SEQUENCE LISTING (15 sequences)

SEQ ID NO: 1 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 2 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 3 - protein, 363 aa, Rhizomucor miehei
MVLKQRANYLGFLIVFFTAFLVEAVPIKRQSNSTVDSLPPLIPSRTSAPSSSPSTTDPEAPAMSRNGPLPSDVETKYGMALNATSYPDSVVQAMSIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 4 - protein, 269 aa, Aspergillus oryzae
EVSQDLFNQFNLFAQYSAAAYCGKNNRAPAGTNITCTGNACPEVEKADATFLYSFEDSGVGDVTGFLALDNTNKLIVLSFRGSRSIENWINNLRFDLKEINDICSGCRGHAGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHPSPEYWIKSGTGVPVTRNDIVKIEGIDATGGNNQPNIPDITAHLWYFGLIGTCL

SEQ ID NO: 5 - protein, 462 aa, Moesziomyces antarcticus
MRVSLRSITSLLAAATAAVLAAPAAETLDRRAALPNPYDDPFYTTPSNIGTFAKGQVIQSRKVPTDIGNANNAASFQLQYRTTNTQNEAVADVATVWIPAKPASPPKIFSYQVYEDATALDCAPSYSYLTGLDQPNKVTAVLDTPIIIGWALQQGYYVVSSDHEGFKAAFIAGYEEGMAILDGIRALKNYQNLPSDSKVALEGYSGGAHATVWATSLAESYAPELNIVGASHGGTPVSAKDTFTFLNGGPFAGFALAGVSGLSLAHPDMESFIEARLNAKGQRTLKQIRGRGFCLPQVVLTYPFLNVFSLVNDTNLLNEAPIASILKQETVVQAEASYTVSVPKFPRFIWHAIPDEIVPYQPAATYVKEQCAKGANINFSPYPIAEHLTAEIFGLVPSLWFIKQAFDGTTPKVICGTPIPAIAGITTPSADQVLGSDLANQLRSLDGKQSAFGKPFGPITPP

SEQ ID NO: 6 - protein, 342 aa, Moesziomyces antarcticus
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 7 - protein, 291 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 8 - protein, 291 aa, Thermomyces lanuginosus
MRSSLVLFFVSAWTALASPIRREVSVDLFNQFNLFAQYSAAAYCGKNNRAPAGTNITCTANACPEVEKADATFLYSFEDSGVGDVTGFLALDNTNKLIVLSFRGSRSIENWITNLNFELKEINDICSGCRGHAGFTSSWRSVADTLRQKVEDAVREHPDYRVVFTGHSLGGALATVAGADLRGNKYDIDVFSYGAPRVGNRAFAEFLTVQTGGTLYRITHTNDIVPRLPPREFGYSHSSPEYWIKSGTLVPVRRRDIVKIEGIDATGGNNQPNIPSITAHLWYFGLIGTCL

SEQ ID NO: 9 - protein, 425 aa, Palaeococcus ferrophilus
MRKYIAVALAIAMFAGLFVVPTMAERPEMVRVVVHIDRSSFNPAGVLNLGGHVVYQFRLIDAAVVEVPSTAVGRLKRLPGVEKVEFDHQAVILKGKPSGAGKPKPSQPPQEVPWGIERVKAPSVWSTTDGSSNGVIQVAILDTGIDYDHPDLAANLAWGVSTLRGRVSTKPKDYRDQNGHGTHVAGTIAALNNDIGVVGVAPGVQIYAIRVLDASGRGSYSDIAIGIEQAILGPDGVADKDGDGIIAGDPDDDAAEVISMSLGGSADDSYLHDMIIQAYNAGIVIVAASGNEGASSPSYPAAYPEVIAVGASDINDNIASFSNRQPEVSAPGVDVLSTYPDDTYKTLSGTSMATPHVSGVVALIQAAHFNKYGTILPVGTFDDMSKNTVRGILHITADDLGSPGWDVDYGYGIVRADLAVQAALG

SEQ ID NO: 10 - protein, 424 aa, Thermococcus litoralis
MEFNKVFSLLLVFVVLGATAGIVGAVSAEKVRVIITIDKDFNENSVFALGGNVVARGKVFPIVIAELSPRAVERLKNAKGVVRVEYDAEVQVLKGKSPGAGKPKPSQPAQTIPWGIERIKAPDVWSITDGSSSGVIEVAILDTGIDYDHPDLAANLAWGVSVLRGKVSTKPKDYKDQNGHGTHVAGTVAALNNDIGVVGVAPAVEIYAVRVLDASGRGSYSDIILGIEQALLGPDGVLDSDGDGIIVGDPDDDAAEVISMSLGGLSDVQAFHDAIIEAYNYGVVIVAASGNEGASSPSYPAAYPEVIAVGATDVNDQVPWWSNRGVEVSAPGVDVLSTYPDDSYETLSGTSMATPHVSGVVALIQAAYYNKYGSVLPVGTFDDNTMSTVRGILHITADDLGSSGWDADYGYGIVRADLAVQAVN

SEQ ID NO: 11 - protein, 422 aa, Thermococcus thioreducens
MGRKDITIALVALIVLSLLGVPATAEKPELVRVIVHVDRGHFNTADVATIGGHVVYQFKLIDAVVVEVPSTAVGRLKKLPGVKMVEFDHKARILAGPPSWLGGGQPSQQIPWGISRVRAPDVWGITDGSGGVIEVAVLDTGVDYDHPDLAGNIAWCVSTLRGRVTTNPAQCKDQNGHGTHVIGTIAALNNDIGVVGVAPGVEIYSIRVLDASGSGSYSDIAIGIEQALLGPDGILDKDGDGIIVGDPDDDAAEVISMSLGGPTDDQYLHDMIITAYNYGVVIVAASGNEGASSPSYPAAYPEVIAVGASDVNDQIASWSNRQPEVSAPGVDILSTYPDDTYETLSGTSMATPHVSGVVALIQAAYYNKYGKVLPVGTFDDMGTNTVRGILHVTADDLGDAGWDIYYGYGIVRADLAVQAAIG

SEQ ID NO: 12 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 13 - protein, 591 aa, Talaromyces emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 14 - protein, 556 aa, Trametes cingulate
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 15 - protein, 583 aa, Artificial sequence
  Synthetic construct
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

(19) United States
(12) Patent Application Publication
(10) Pub. No.: US 2021/0269833 A1
(43) Pub. Date: Sep. 2, 2021

(54) PROCESSES FOR PRODUCING FERMENTATION PRODUCTS

(71) Applicant: NOVOZYMES A/S, Bagsvaerd (DK)

(72) Inventors: Angela Shows, Raleigh, NC (US); Alan Jay House, Cary, NC (US); Guillermo Coward Kelly, Wake Forest, NC (US); James LaVigne, Wake Forest, NC (US)

(73) Assignee: NOVOZYMES A/S, Bagsvaerd (DK)

(21) Appl. No.: 17/259,214

(22) PCT Filed: Jul. 11, 2019
(86) PCT No.: PCT/US2019/041280

§ 371 Date: Jan. 11, 2021

Related U.S. Application Data
(60) Provisional application No. 62/696,515, filed on Jul. 11, 2018.

(51) Int. Cl. C12P 7/10

(52) U.S. Cl. CPC C12P 7/10; C12R 2001/85; C12Y 301/01003; C12Y 302/01001

(57) ABSTRACT

The present disclosure relates to processes for producing fermentation products from starch-containing material, wherein a triacylglyceride lipase (e.g., a thermostable triacylglyceride lipase) is present and/or added during liquefaction, pre-saccharification, saccharification, fermentation, simultaneous saccharification and fermentation, or any combination thereof, to increase enzymatically accessible starch, for example by reducing starch retrogradation, and/or increase fermentation product yield, such as ethanol yield. The disclosure also relates to the use of a triacylglyceride lipase in processes of the disclosure, for example, to increase enzymatically accessible starch and/or fermentation product yield, such as ethanol yield.

### CROSS-REFERENCE TO RELATED APPLICATIONS

This application claims priority to U.S. Provisional Patent Application No. 62/696,515, filed Jul. 11, 2018, the disclosure of which is incorporated herein by reference in its entirety.

### FIELD OF THE INVENTION

The present disclosure relates to processes for producing fermentation products, especially ethanol, from starch-containing material. The disclosure also relates to use of a triacylglycerol lipase (e.g., thermostable) during liquefaction and/or saccharification, fermentation, or simultaneous saccharification and fermentation in a fermentation product production process of the disclosure to increase enzymatically accessible starch, for example by reducing starch retrogradation, and/or increase fermentation product yield, such as especially ethanol.

### Reference to a Sequence Listing

This application contains a Sequence Listing in computer readable form. The computer readable form is incorporated herein by reference.

### BACKGROUND OF THE INVENTION

Production of fermentation products, such as ethanol, from starch-containing material is well-known in the art. Industrially two different kinds of processes are used today. The most commonly used process, often referred to as a “conventional process”, includes liquefying gelatinized starch at high temperature using typically a bacterial alpha-amylase, followed by simultaneous saccharification and fermentation carried out in the presence of a glucoamylase and a fermentation organism. Another well-known process, often referred to as a “raw starch hydrolysis”-process (RSH process), includes simultaneously saccharifying and fermenting granular starch below the initial gelatinization temperature typically in the presence of at least a glucoamylase.

Despite significant improvement of fermentation product production processes over the past decade a significant amount of residual starch material is not converted into the desired fermentation product, such as ethanol.

Therefore, there is still a desire and need for providing processes for producing fermentation products, such as ethanol, from starch-containing material that can provide a higher amount of enzymatically accessible starch and/or fermentation product yield, or other advantages, compared to conventional processes.

### SUMMARY OF THE INVENTION

The object of the present disclosure is to provide processes for producing fermentation products, such as ethanol, from starch-containing material that can provide an increased amount of enzymatically accessible starch and/or fermentation product yield, or other advantages, compared to conventional processes.

In an aspect the present disclosure relates to a method of increasing enzymatically accessible starch, for example by reducing starch retrogradation, and/or increasing fermentation product yield, such as especially ethanol, during a fermentation product production process, wherein a triacylglycerol lipase is present and/or added before or during the liquefaction step, the saccharification step, the fermentation step, or simultaneous saccharification and fermentation step, of the fermentation product production process. In some embodiments, the triacylglycerol lipase is present and/or added before or during the liquefaction step and present and/or added during the saccharification step, fermentation step, or simultaneous saccharification and fermentation step.

In an aspect the disclosure relates to processes of producing fermentation products, comprising: (a) liquefying a starch-containing material using an alpha-amylase; (b) saccharifying the liquified starch-containing material using a carbohydrate-source generating enzyme to form fermentable sugars; and (c) fermenting the fermentable sugars using a fermenting organism to product the fermentation product, wherein a triacylglycerol lipase is present and/or added before or during liquefying step (a), saccharifying step (b), fermenting step (c), or simultaneous saccharification and fermentation. In some embodiments, the triacylglycerol lipase is present and/or added before or during liquefying step (a) and before or during saccharifying step (b), fermenting step (c), or simultaneous saccharification and fermentation.

In preferred embodiments the fermentation production product is ethanol and the enzymatically accessible starch and/or ethanol yield is increased compared to performance of the method in the absence of using a triacylglycerol lipase.

In a preferred embodiment the triacylglycerol lipase is a thermostable triacylglycerol lipase, preferably having a Melting Point (DSC) greater than or equal to about 60° C., such as between 60° C. and 110° C., such as between 65° C. and 95° C., such as between 70° C. and 90° C., such as above 70° C., such as above 72° C., such as above 80° C., such as above 85° C., such as above 90° C., such as above 92° C., such as above 94° C., such as above 96° C., such as above 98° C., such as above 100° C.

Examples of thermostable triacylglycerol lipases of use herein include: (i) the triacylglycerol lipase shown in SEQ ID NO: 3 herein derived from a strain of Rhizomucor miehei; or a polypeptide having triacylglycerol lipase activity, having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3 herein; (ii) the triacylglycerol lipase shown in SEQ ID NO: 4 derived from a strain of Aspergillus oryzae; or a polypeptide having triacylglycerol lipase activity, having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 4 herein; (iii) the triacylglycerol lipase shown in SEQ ID NO: 5 derived from a strain of Moesziomyces antarcticus; or a polypeptide having triacylglycerol lipase activity, having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 5 herein; (iv) the triacylglycerol lipase shown in SEQ ID NO: 6 derived from a strain of Moesziomyces antarcticus or a polypeptide having triacylglycerol lipase activity, having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 6 herein; (v) the triacylglycerol lipase shown in SEQ ID NO: 7 derived from a strain of Thermomyces lanuginosus or a polypeptide having triacylglycerol lipase activity, having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO:7 herein; and (vi) the triacylglycerol lipase shown in SEQ ID NO: 8 derived from a strain of Thermomyces lanuginosus or a polypeptide having triacylglycerol lipase activity, having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 8 herein.

Other enzymes such as endoglucanase, hemicellulases (e.g., xylanases, preferably a thermostable xylanase), carbohydrate source generating enzymes (e.g., glucoamylase, preferably a thermostable glucoamylase), proteases, pullulanases and phytases may also be used in the processes of the present disclosure.

### BRIEF DESCRIPTION OF THE FIGURES

FIG. 1 is a graph depicting the results from primary screening at 20% dry solids (DS) at a 24 hr time point, showing that Rm TG lipase and Ao TG lipase improved ethanol titers compared to the control treatment lacking a TG lipase.

FIG. 2A is a graph depicting the results from secondary screening at 32% DS at a 24 hr time point, showing the affect of TG lipases on ethanol titers compared to the control treatment.

FIG. 2B is a graph depicting the results from secondary screening at 32% DS at a 60 hr time point, showing the affect of TG lipases on ethanol titers compared to the control treatment.

FIG. 3 is a graph depicting the results from incubating liquified mash samples with Alpha-Amylase and Glucoamylase, showing an increase in the amount of enzymatically accessible starch after TG lipase treatment for all lipases tested.

### SOME DEFINITIONS

Unless defined otherwise or clearly indicated by context, all technical and scientific terms used herein have the same meaning as commonly understood by one of ordinary skill in the art.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-Amylases (alpha-1,4-glucan-4-glucanohydrolases, EC 3.2.1.1) are a group of enzymes, which catalyze the hydrolysis of starch and other linear and branched 1,4-glucosidic oligo- and polysaccharides.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Catalytic domain: The term “catalytic domain” means the region of an enzyme containing the catalytic machinery of the enzyme.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase: The term “cellulolytic enzyme” or “cellulase” means one or more (e.g., several) enzymes that hydrolyze a cellulosic-containing material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N21 filter paper, microcrystalline cellulose, bacterial cellulose, algal cellulose, cotton, pretreated lignocellulose, etc. The most common total cellulolytic activity assay is the filter paper assay using Whatman N21 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic-containing material by cellulolytic enzyme(s) under the following conditions: 1-50 mg of cellulolytic enzyme protein/g of cellulose in pretreated corn stover (PCS) (or other pretreated cellulosic-containing material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc., Hercules, Calif., USA).

Endoglucanase: The term “endoglucanase” means a 4-(1,3; 1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Fragment: The term “fragment” means a polypeptide having one or more (e.g., several) amino acids absent from the amino and/or carboxyl terminus of a mature polypeptide; wherein the fragment has triacylglycerol activity.

Glucoamylases (glucan 1,4-alpha-glucosidase, EC 3.2.1.3) are a group of enzymes, which catalyze the hydrolysis of terminal (1→4)-linked a-D-glucose residues successively from non-reducing ends of the chains with release of beta-D-glucose.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Host cell: The term “host cell” means any cell type that is susceptible to transformation, transfection, transduction, and the like with a nucleic acid construct or expression vector comprising a polynucleotide described herein (e.g., a polynucleotide encoding a peptide or amino acid transporter, or regulator thereof). The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide having biological activity that is in its final form following translation and any post-translational modifications, such as N-terminal processing, C-terminal truncation, glycosylation, phosphorylation, etc. In one embodiment, the mature polypeptide is amino acids 95 to 363 of SEQ ID NO: 3, as amino acids 1 to 24 of SEQ I D NO: 3 are predicted to be a signal peptide and amino acids 25-94 are a propeptide. In one embodiment, the mature polypeptide is amino acids 22 to 462 of SEQ ID NO: 5, as amino acids 1 to 21 of SEQ ID NO: 5 are predicted to be a signal peptide. In one embodiment, the mature polypeptide is amino acids 20 to 342 of SEQ ID NO: 5, as amino acids 1 to 19 of SEQ ID NO: 5 are predicted to be a signal peptide. In one embodiment, the mature polypeptide is amino acids 18 to 291 of SEQ ID NO: 7, as amino acids 1 to 17 of SEQ I D NO: 7 are predicted to be a signal peptide. In one embodiment, the mature polypeptide is amino acids 18 to 291 of SEQ ID NO: 8, as amino acids 1 to 17 of SEQ I D NO: 8 are predicted to be a signal peptide. It is known in the art that a host cell may produce a mixture of two of more different mature polypeptides (i.e., with a different C-terminal and/or N-terminal amino acid) expressed by the same polynucleotide.

Protease: The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 223: 1-5 (1994); Eur. J. Biochem. 232: 1-6 (1995); Eur. J. Biochem. 237: 1-5 (1996); Eur. J. Biochem. 250: 1-6 (1997); and Eur. J. Biochem. 264: 610-650 (1999); respectively. The term “subtilases” refer to a sub-group of serine protease according to Siezen et al., 1991, Protein Engng. 4: 719-737 and Siezen et al., 1997, Protein Science 6: 501-523.

Proteases are classified on the basis of their catalytic mechanism into the following groups: Serine proteases (S), Cysteine proteases (C), Aspartic proteases (A), Metalloproteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

Polypeptides having protease activity, or proteases, are sometimes also designated peptidases, proteinases, peptide hydrolases, or proteolytic enzymes. Proteases may be of the exo-type (exopeptidases) that hydrolyse peptides starting at either end thereof, or of the endo-type that act internally in polypeptide chains (endopeptidases).

In particular embodiments, the proteases for use in the processes of the invention are selected from the group consisting of:
(a) proteases belonging to the EC 3.4.24 metalloendopeptidases;
(b) metalloproteases belonging to the M group of the above Handbook;
(c) metalloproteases not yet assigned to clans (designation: Clan MX), or belonging to either one of clans MA, MB, MC, MD, ME, MF, MG, MH (as defined at pp. 989-991 of the above Handbook);
(d) other families of metalloproteases (as defined at pp. 1448-1452 of the above Handbook);
(e) metalloproteases with a HEXXH motif;
(f) metalloproteases with an HEFTH motif;
(g) metalloproteases belonging to either one of families M3, M26, M27, M32, M34, M35, M36, M41, M43, or M47 (as defined at pp. 1448-1452 of the above Handbook); and
(h) metalloproteases belonging to family M35 (as defined at pp. 1492-1495 of the above Handbook).

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, J. Mol. Biol. 1970, 48, 443-453) as implemented in the Needle program of the EMBOSS package (EMBOSS: The European Molecular Biology Open Software Suite, Rice et al., Trends Genet 2000, 16, 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of the Referenced Sequence−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Referenced Sequence−Total Number of Gaps in Alignment)

Signal peptide: The term “signal peptide” is defined herein as a peptide linked (fused) in frame to the amino terminus of a polypeptide having biological activity and directs the polypeptide into the cell's secretory pathway.

Triacylglycerol activity: The term “triacylglycerol activity” means the activity that catalyzes the reaction: triacylglycerol+H₂O=diacylglycerol+a carboxylate. Triacylglycerol activity may be determined using a triacylglycerol activity assay (see, for example, in Wilton, Biochem J 1991 May 15; 276 (Pt I):129-33, which is incorporated herein by reference. An enzyme having “triacylglycerol activity” may belong to EC 3.1.1.3.

Variant: The term “variant” means a polypeptide having triacylglyceride activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. A variant may include substitution, insertion, and/or deletion of up to 20 amino acids, e.g., 1, 2, 3, 4, 5, 6, 7, 8, 9, 10, 11, 12, 13, 14, 15, 16, 17, 18, 19, or 20. Whether an amino acid change results in a catalytically active triacylglycerol lipase polypeptide can readily be determined by assaying for triacylglycerol lipase activity, as described for example, in Wilton, Biochem J 1991 May 15; 276 (Pt I):129-33.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value, and can include a range of plus or minus two standard deviations around the stated value.

Likewise, reference to a gene or polypeptide that is “derived from” another gene or polypeptide X, includes the gene or polypeptide X.

As used herein and in the appended claims, the singular forms “a,” “or,” and “the” include plural referents unless the context clearly dictates otherwise.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

### DETAILED DESCRIPTION OF THE INVENTION

The object of the present disclosure is to provide processes for producing fermentation products, such as ethanol, from starch-containing material that can increase enzymatically accessible starch, or provide other advantages, compared to conventional processes.

The present disclosure relates to the use of a triacylglycerol lipase during the liquefaction step and/or saccharification, fermentation, or simultaneous saccharification and fermentation step in a fermentation product production process. The use of triacylglycerol lipase increases enzymatically accessible starch during fermentation, for example by reducing starch retrogradation, resulting in a higher fermentation product yield, such as especially ethanol.

### I. Methods of Increasing Enzymatically Accessible Starch/Fermentation Product Yield

The inventors have found that increased enzymatically accessible starch and fermentation product yield, such as especially ethanol yield, are obtained when the liquefaction and/or saccharification, fermentation, or simultaneous saccharification and fermentation steps of a fermentation product production process are carried out in the presence of a triacylglycerol lipase (e.g., thermostable). (see Examples).

Accordingly, in the first aspect the present disclosure relates to a method of increasing enzymatically accessible starch, for example by reducing starch retrogradation, and/or fermentation product yield, during a fermentation product production process, wherein a triacylglycerol lipase is present and/or added before or during a liquefaction step and/or a saccharification step, a fermentation step, or simultaneous saccharification and fermentation step of the fermentation product production process.

As used herein, the phrase “present and/or added before or during” a particular step of a fermentation product production process means that an amount of an enzyme (e.g., triacylglycerol lipase) is added before or during the particular step of the fermentation product production process.

In a preferred embodiment the fermentation product is ethanol and the method increases enzymatically accessible starch, for example by reducing starch retrogradation, resulting in increases in ethanol yield.

In a preferred embodiment the triacylglycerol lipase is a fungal triacylglycerol lipase.

In a preferred embodiment the triacylglycerol lipase, e.g., one derived from a strain of Rhizomucor, for example Rhizomucor miehei, is the mature part of the sequence shown as SEQ ID NO: 3, or a sequence having a sequence identity thereto of at least 60%, at least 65%, at least 70%, at least 75%, at least 80%, at least 85%, at least 90%, at least 95%, at least 96%, at least 97%, at least 98%, at least 99%, to the mature part of the sequence shown in SEQ ID NO: 3.

In an embodiment the triacylglycerol lipase shown in SEQ ID NO: 3 is present and/or added during liquefaction, pre-saccharification, saccharification, fermentation, and/or simultaneous saccharification and fermentation.

In a preferred embodiment the triacylglycerol lipase, e.g., one derived from a strain of Aspergillus, for example Aspergillus oryzae, is the mature part of the sequence shown as SEQ ID NO: 4, or a sequence having a sequence identity thereto of at least 60%, at least 65%, at least 70%, at least 75%, at least 80%, at least 85%, at least 90%, at least 95%, at least 96%, at least 97%, at least 98%, at least 99%, to the mature part of the sequence shown in SEQ ID NO: 4.

In an embodiment the triacylglycerol lipase shown in SEQ ID NO: 4 is present and/or added during liquefaction pre-saccharification, saccharification, fermentation, and/or simultaneous saccharification and fermentation.

In a preferred embodiment the triacylglycerol lipase, e.g., one derived from a strain of Moesziomyces, for example Moesziomyces antarcticus, is the mature part of the sequence shown as SEQ ID NO: 5, or a sequence having a sequence identity thereto of at least 60%, at least 65%, at least 70%, at least 75%, at least 80%, at least 85%, at least 90%, at least 95%, at least 96%, at least 97%, at least 98%, at least 99%, to the mature part of the sequence shown in SEQ ID NO: 5, or the mature part of the sequence shown as SEQ ID NO: 6, or a sequence having a sequence identity thereto of at least 60%, at least 65%, at least 70%, at least 75%, at least 80%, at least 85%, at least 90%, at least 95%, at least 96%, at least 97%, at least 98%, at least 99%, to the mature part of the sequence shown in SEQ ID NO: 6.

In an embodiment the triacylglycerol lipase shown in SEQ ID NO: 5 is present and/or added during liquefaction pre-saccharification, saccharification, fermentation, and/or simultaneous saccharification and fermentation. In an embodiment the triacylglycerol lipase shown in SEQ ID NO: 6 is present and/or added during liquefaction pre-saccharification, saccharification, fermentation, and/or simultaneous saccharification and fermentation.

In a preferred embodiment the triacylglycerol lipase, e.g., one derived from a strain of Thermomyces, for example Thermomyces lanuginosus, is the mature part of the sequence shown as SEQ ID NO: 7, or a sequence having a sequence identity thereto of at least 60%, at least 65%, at least 70%, at least 75%, at least 80%, at least 85%, at least 90%, at least 95%, at least 96%, at least 97%, at least 98%, at least 99%, to the mature part of the sequence shown in SEQ ID NO: 7, or the mature part of the sequence shown as SEQ ID NO: 8, or a sequence having a sequence identity thereto of at least 60%, at least 65%, at least 70%, at least 75%, at least 80%, at least 85%, at least 90%, at least 95%, at least 96%, at least 97%, at least 98%, at least 99%, to the mature part of the sequence shown in SEQ ID NO: 8.

In an embodiment the triacylglycerol lipase shown in SEQ ID NO: 7 is present and/or added during liquefaction. In an embodiment the triacylglycerol lipase shown in SEQ ID NO: 8 is present and/or added during liquefaction, pre-saccharification, saccharification, fermentation, and/or simultaneous saccharification and fermentation.

The liquefaction is carried out by liquefying a starch-containing material at a temperature above the initial gelatinization temperature using an alpha-amylase, e.g., a bacterial alpha-amylase and the triacylglycerol lipase (e.g., fungal triacylglycerol lipase).

In an embodiment, the triacylglycerol lipase has a Melting Point (DSC) of at least about 65° C. In an embodiment, the triacylglycerol lipase has a Melting Point (DSC) of at least about 70° C. In an embodiment, the triacylglycerol lipase has a Melting Point (DSC) of at least about 73° C. In an embodiment, the triacylglycerol lipase has a Melting Point (DSC) of at least about 86° C. In an embodiment, the triacylglycerol lipase has a Melting Point (DSC) of at least about 90° C.

Examples of suitable and preferred enzymes can be found below.

### II. Process of Producing Fermentation Products

In another aspect the present disclosure relates to processes of producing fermentation products, comprising: (a) liquefying a starch-containing material using an alpha-amylase; (b) saccharifying the liquified starch-containing material using a carbohydrate-source generating enzyme to form fermentable sugars; and (c) fermenting the fermentable sugars using a fermenting organism to product the fermentation product, wherein a triacylglycerol lipase is added before or during liquefying step (a) and/or saccharifying step (b), fermenting step (c), or simultaneous saccharification and fermentation.

Liquefaction step (a), saccharification step (b) and fermentation step (c) are carried out sequentially, though saccharification step (b) and fermentation step (c) may be carried out simultaneously (SSF).

### A. Liquefaction Step (a)

Generally the starch-containing material in step (a) may contain 10-55 wt.-% dry solids (DS), preferably 25-45 wt.-% dry solids, more preferably 30-40% dry solids.

The alpha-amylase and/or the triacylglycerol lipase may be added before and/or during liquefaction step (a) optionally with a protease and/or a glucoamylase. Other enzymes such as a pullulanase, endoglucanase, hemicellulase (e.g., xylanase), phospholipase C, and phytase may also be present and/or added in liquefaction.

In an embodiment the pH in step (a) may be between 4 and 7, such as pH 4.5-6.5, pH 5.0-6.5, pH 5.0-6.0, pH 5.2-6.2, or about 5.2, about 5.4, about 5.6, or about 5.8.

Step (a) may be carried out at as a liquefaction step at a temperature above the initial gelatinization temperature.

The term “initial gelatinization temperature” means the lowest temperature at which gelatinization of the starch commences. Starch heated in water begins to gelatinize between 50° C. and 75° C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this disclosure the initial gelatinization temperature of a given starch-containing material is the temperature at which birefringence is lost in 5% of the starch granules using the method described by Gorinstein. S. and Lii. C, Starch/Starke, Vol. 44 (12) pp. 461-466 (1992).

In an embodiment step (a) is carried out at a temperature between 60° C. and 100° C. In an embodiment step (a) is carried out at a temperature between 70° C. and 100° C. In an embodiment step (a) is carried about at a temperature between 80-90° C. In an embodiment step (a) is carried about at a temperature of about 82° C. In an embodiment step (a) is carried about at a temperature of about 83° C. In an embodiment step (a) is carried about at a temperature of about 84° C. In an embodiment step (a) is carried about at a temperature of about 86° C. In an embodiment step (a) is carried about at a temperature of about 87° C. In an embodiment step (a) is carried about at a temperature of about 88° C. In an embodiment step (a) is carried about at a temperature of about 90° C.

In an embodiment a jet-cooking step may be carried out before in step (a). Jet-cooking may be carried out at a temperature between 95-140° C. for about 1-15 minutes, preferably for about 3-10 minutes, especially about 5 minutes.

In an embodiment a process of the disclosure further comprises, before step (a), and optional jet-cooking step, the steps of: i) reducing the particle size of the starch-containing material, preferably by dry milling; and ii) forming a slurry comprising the starch-containing material and water.

The starch-containing starting material, such as whole grains, may be reduced in particle size, e.g., by milling, in order to open up the structure, to increase the surface area and allowing for further processing. Generally there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry and wet millings are well known in the art of starch processing. According to the present disclosure dry milling is preferred. In an embodiment the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen. In another embodiment at least 50%, preferably at least 70%, more preferably at least 80%, especially at least 90% of the starch-containing material fit through a sieve with #6 screen.

The aqueous slurry may contain from 10-55 w/w-% dry solids (DS), preferably 25-45 w/w-% dry solids (DS), more preferably 30-40 w/w-% dry solids (DS) of starch-containing material.

The slurry may be heated to above the initial gelatinization temperature, preferably to between 70-95° C., such as between 80-90° C., between pH 5.0-7.0, preferably between 5.0 and 6.0, for 30 minutes to 5 hours, such as around 2 hours.

In an embodiment liquefaction step a) is carried out for 0.5-5 hours at a temperature from 70-95° C. at a pH from 4-6.

In a preferred embodiment liquefaction step a) is carried out for 0.5-3 hours at a temperature from 80-90° C. at a pH from 4-6.

The alpha-amylase and/or triacylglycerol lipase and optionally a protease and/or a glucoamylase may initially be added to the aqueous slurry to initiate liquefaction (thinning). In an embodiment only a portion of the enzymes (e.g., about ¼, about ⅓, about ½, etc.) is added to the aqueous slurry, while the rest of the enzymes (e.g., about ¾, about ⅔, about ½, etc. are added during liquefaction step a).

The aqueous slurry may in an embodiment be jet-cooked to further gelatinize the slurry before being subjected to liquefaction in step a). The jet-cooking may be carried out at a temperature between 95-160° C., such as between 110-145° C., preferably 120-140° C., such as 125-135° C., preferably around 130° C. for about 1-15 minutes, preferably for about 3-10 minutes, especially about 5 minutes.

A non-exhaustive list of alpha-amylases used in liquefaction can be found below in the “Alpha-Amylases” section. Examples of suitable proteases used in liquefaction include any protease described in the “Proteases” section. Examples of suitable triacylglyceride lipases used in liquefaction include any triacylgyceride lipase described in the “Triacylglyceride Lipases” section. Examples of suitable glucoamylases used in liquefaction include any glucoamylase found in the “Glucoamylases in liquefaction” section.

### Alpha-Amylases

The alpha-amylase used in step (a) may be any alpha-amylase, but is preferably a bacterial alpha-amylase. In a preferred embodiment the bacterial alpha-amylase is derived from the genus Bacillus. A preferred bacterial alpha-amylase may be derived from a strain of Bacillus stearothermophilus, and may be a variant of a Bacillus stearothermophilus alpha-amylase, such as the one shown as SEQ ID NO: 1. Bacillus stearothermophilus alpha-amylases are typically truncated naturally during production. In particular the alpha-amylase may be a truncated Bacillus stearothermophilus alpha-amylase having from 485-495 amino acids, such as one being around 491 amino acids long (SEQ ID NO: 1).

According to the present disclosure the Bacillus stearothermophilus alpha-amylase may be the one shown as SEQ ID NO: 1 or one having a sequence identity thereto of at least 60%, at least 70%, at least 80%, at least 90%, at least 95%, at least 96%, at least 97%, at least 98%, at least 99%.

In an embodiment the bacterial alpha-amylase may be selected from the group of Bacillus stearothermophilus alpha-amylase variants comprising a deletion of one or two amino acids at any of positions R179, G180, I181 and/or G182, preferably the double deletion disclosed in WO 96/23873—see, e.g., page 20, lines 1-10 (hereby incorporated by reference), preferably corresponding to deletion of positions I181+G182 compared to the amino acid sequence of Bacillus stearothermophilus alpha-amylase set forth as SEQ ID NO: 3 disclosed in WO 99/19467 or SEQ ID NO: 1 herein or the deletion of amino acids R179+G180 using SEQ ID NO: 1 herein for numbering.

In a preferred embodiment the Bacillus stearothermophilus alpha-amylase variant comprises one of the following set of mutations:—R179*+G180*; —I181*+G182*; —I181*+G182*+N193F; preferably—I181*+G182*+N193F+E129V+K177L+R179E; —I181*+G182*+N193F+V59A+Q89R+E129V+K177L+R179E+H208Y+K220P+N224L+Q254S; I181*+G182*+N193F+V59A Q89R+E129V+K177L+R179E+Q254S+M284V; and —I181*+G182*+N193F+E129V+K177L+R179E+K220P+N224L+S242Q+Q254S (using SEQ ID NO: 1 for numbering).

In an embodiment the Bacillus stearothermophilus alpha-amylase variant has a sequence identity to SEQ ID NO: 1 of at least 60%, at least 70%, at least 80%, at least 90%, at least 95%, at least 96%, at least 97%, at least 98%, at least 99%, but less than 100%.

In an embodiment the Bacillus stearothermophilus alpha-amylase variant has from 1-12 mutations, such as 1, 2, 3, 4, 5, 6, 7, 8, 9, 10, 11, or 12 mutations, compared to the parent alpha-amylase, especially the alpha-amylase shown as SEQ ID NO: 1.

Commercially available bacterial alpha-amylase products and products containing alpha-amylases include TERMAMYL™ SC, LIQUOZYME™ SC, LIQUOZYME™ LpH, AVANTEC™, AVANTEC™ AMP, BAN (Novozymes A/S, Denmark) DEX-LO™, SPEZYME™ XTRA, SPEZYME™ AA, SPEZYME™ FRED-L, SPEZYME™ ALPHA, GC358™, SPEZYME™ RSL, SPEZYME™ HPA and SPEZYME™ DELTA AA (from DuPont, USA), FUELZYME®, FUELZYME®-LF (BASF/Verenium, USA).

A bacterial alpha-amylase may be added in step (a) in an amount well-known in the art.

In an embodiment the bacterial alpha-amylase, e.g., Bacillus alpha-amylase, such as especially Bacillus stearothermophilus alpha-amylase, or variant thereof, is dosed in liquefaction in a concentration between 0.01-10 KNU-A/g DS, e.g., between 0.02 and 5 KNU-A/g DS, such as 0.03 and 3 KNU-A, preferably 0.04 and 2 KNU-A/g DS, such as especially 0.01 and 2 KNU-A/g DS. In an embodiment the bacterial alpha-amylase, e.g., Bacillus alpha-amylase, such as especially Bacillus stearothermophilus alpha-amylases, or variant thereof, is dosed to liquefaction in a concentration of between 0.0001-1 mg EP (Enzyme Protein)/g DS, e.g., 0.0005-0.5 mg EP/g DS, such as 0.001-0.1 mg EP/g DS.

### Triacylglyceride Lipases

According to the present disclosure triacylglyceride lipase (e.g., fungal triacylglyceride lipase), preferably a thermostable triacylglyceride lipase having a Melting Point (DSC) of at least about 65° C., is added before or during liquefying step a) and/or saccharifying step (b), fermenting step (c) or simultaneous saccharification and fermentation.

The thermostability of a triacylglyceride lipase may be determined as described in the Materials & Methods section herein.

In an embodiment the triacylglyceride lipase has a Melting Point (DSC) of greater than or equal to about 60° C., such as between 60° C. and 110° C., such as between 65° C. and 95° C., such as between 70° C. and 90° C., such as above 70° C., such as above 72° C., such as above 80° C., such as above 85° C., such as above 90° C., such as above 92° C., such as above 94° C., such as above 96° C., such as above 98° C., such as above 100° C.

In a preferred embodiment the triacylglyceride lipase has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3 herein, preferably derived from a strain of the genus Rhizomucor, such as a strain of Rhizomucor miehei.

In an embodiment the triacylglyceride lipase comprises or consists of the amino acid sequence of SEQ ID NO: 3, or an allelic variant thereof; or is a fragment thereof having triacylglycerol lipase activity. In another embodiment, the triacylglyceride lipase comprises or consists of the mature polypeptide of SEQ ID NO: 3, or a variant of the mature polypeptide of SEQ ID NO: 3 comprising a substitution, deletion, and/or inseration at one or more positions. In another embodiment, the triacylglyceride lipase comprises or consists of amino acids 1 to 363 of SEQ ID NO: 3. In another embodiment, the triacylglyceride lipase comprises or consists of amino acids 25 to 363 of SEQ ID NO: 3. In another embodiment, the triacylglycerol lipase comprises or consists of amino acids 95-363 of SEQ ID NO: 3.

In a preferred embodiment the triacylglyceride lipase has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 4 herein, preferably derived from a strain of the genus Aspergillus, such as a strain of Aspergillus oryzae.

In an embodiment the triacylglyceride lipase comprises or consists of the amino acid sequence of SEQ ID NO: 4, or an allelic variant thereof; or is a fragment thereof having triacylglycerol lipase activity. In another embodiment, the triacylglyceride lipase comprises or consists of the mature polypeptide of SEQ ID NO: 4, or a variant of the mature polypeptide of SEQ ID NO: 4 comprising a substitution, deletion, and/or insertion at one or more positions. In another embodiment, the triacylglyceride lipase comprises or consists of amino acids 1 to 269 of SEQ ID NO: 4.

In a preferred embodiment the triacylglyceride lipase has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 5 or SEQ ID NO: 6 herein, preferably derived from a strain of the genus Moesziomyces, such as a strain of Moesziomyces antarcticus.

In an embodiment the triacylglyceride lipase comprises or consists of the amino acid sequence of SEQ ID NO: 5 or SEQ ID NO: 6, or an allelic variant thereof; or is a fragment thereof having triacylglycerol lipase activity. In another embodiment, the triacylglyceride lipase comprises or consists of the mature polypeptide of SEQ ID NO: 5 or SEQ ID NO: 6, or a variant of the mature polypeptide of SEQ ID NO: 5 or SEQ ID NO: 6 comprising a substitution, deletion, and/or inseration at one or more positions. In another embodiment, the triacylglyceride lipase comprises or consists of amino acids 1 to 342 of SEQ ID NO: 5. In another embodiment, the triacylglyceride lipase comprises or consists of amino acids 20 to 342 of SEQ ID NO: 5. In another embodiment, the triacylglyceride lipase comprises or consists of amino acids 1 to 291 of SEQ ID NO: 6. In another embodiment, the triacylglyceride lipase comprises or consists of amino acids 18-291 of SEQ ID NO: 6.

In a preferred embodiment the triacylglyceride lipase has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 7 or SEQ ID NO: 8 herein, preferably derived from a strain of the genus Thermomyces, such as a strain of Thermomyces lanuginosus.

In an embodiment the triacylglyceride lipase comprises or consists of the amino acid sequence of SEQ ID NO: 7 or SEQ ID NO: 8, or an allelic variant thereof; or is a fragment thereof having triacylglycerol lipase activity. In another embodiment, the triacylglyceride lipase comprises or consists of the mature polypeptide of SEQ ID NO: 7 or SEQ ID NO: 8, or a variant of the mature polypeptide of SEQ ID NO: 7 or SEQ ID NO: 8 comprising a substitution, deletion, and/or inseration at one or more positions. In another embodiment, the triacylglyceride lipase comprises or consists of amino acids 1 to 291 of SEQ ID NO: 7 or SEQ ID NO: 8. In another embodiment, the triacylglyceride lipase comprises or consists of amino acids 18 to 291 of SEQ ID NO: 7 or SEQ ID NO: 8.

A triacylglyceride lipase may be added and/or present in step (a) in an amount effective to increase enzymatically accessible starch and/or fermentation product yield, such as especially ethanol yield, during SSF steps (b) and (c) or fermentation step (c).

In an embodiment the triacylglyceride lipase, such as especially Rhizomucor miehei triacylglyceride lipase, or variant thereof, is dosed in liquefaction in a concentration of about 0.1-50,000 μg EP (Enzyme Protein)/g DS, such as 10,000 μg EP (Enzyme Protein)/g DS, or especially such as 5-1000 μg EP/g DS.

In an embodiment the triacylglyceride lipase, such as especially Aspergillus oryzae triacylglyceride lipase, or variant thereof, is dosed in liquefaction in a concentration of about 0.1-50,000 μg EP (Enzyme Protein)/g DS, such as 10,000 μg EP (Enzyme Protein)/g DS, or especially such as 5-1000 μg EP/g DS.

In an embodiment the triacylglyceride lipase, such as especially Moesziomyces antarcticus triacylglyceride lipase, or variant thereof, is dosed in liquefaction in a concentration of about 0.1-50,000 μg EP (Enzyme Protein)/g DS, such as 10,000 μg EP (Enzyme Protein)/g DS, or especially such as 5-1000 μg EP/g DS.

In an embodiment the triacylglyceride lipase, such as especially Thermomyces lanuginosus triacylglyceride lipase, or variant thereof, is dosed in liquefaction in a concentration of about 0.1-50,000 μg EP (Enzyme Protein)/g DS, such as 10,000 μg EP (Enzyme Protein)/g DS, or especially such as 5-1000 μg EP/g DS.

Optionally, an endoglucanase (e.g., thermostable endoglucanase), hemicellulase (e.g., xylanase, preferably a thermostable xylanase), a phospholipase C (e.g., a thermostable phospholipase C), a protease, a carbohydrate-source generating enzyme, (e.g., glucoamylase, preferably a thermostable glucoamylase), a pullulanase, and/or a phytase may be present and/or added during liquefaction step (a). The enzymes may be added individually or as one or more blend compositions. In some embodiments, liquefaction step (a) is carried out in the absence of a protease. In some embodiments, liquefaction step (a) is carried out in the absence of a phospholipase C. In some embodiments, a phospholipase C is not present and/or added in liquefaction step (a).

### Proteases

In the processes described herein, a protease may optionally be present and/or added in slurry and/or liquefaction together with alpha-amylase, triacylglycerol lipase, and an optional glucoamylase, phospholipase C, xylanase, endoglucanase, phytase, and/or pullulanase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In some embodiments, the fermenting organism comprises a heterologous polynucleotide encoding a protease, for example, as described in U.S. Provisional Patent No. 62/514,636 filed Jun. 2, 2017, the content of which is hereby incorporated by reference. Any protease described or referenced herein is contemplated for expression in the fermenting organism.

The protease may be any protease that is suitable for the host cells and/or the methods described herein, such as a naturally occurring protease or a variant thereof that retains protease activity.

In some embodiments, the fermenting organism comprising a heterologous polynucleotide encoding a protease has an increased level of protease activity compared to the host cells without the heterologous polynucleotide encoding the protease, when cultivated under the same conditions. In some embodiments, the fermenting organism has an increased level of protease activity of at least 5%, e.g., at least 10%, at least 15%, at least 20%, at least 25%, at least 50%, at least 100%, at least 150%, at least 200%, at least 300%, or at 500% compared to the fermenting organism without the heterologous polynucleotide encoding the protease, when cultivated under the same conditions.

Exemplary proteases that can be used with the host cells and/or the methods described herein include archae, bacterial, yeast, or filamentous fungal proteases, e.g., derived from any of the microorganisms described or referenced herein.

In one embodiment, the thermostable protease used according to a process described herein is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein).

In one embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the Protease 196 variant or Protease Pfu.

There are no limitations on the origin of the protease used in a process described herein as long as it fulfills the thermostability properties defined below.

In one embodiment the protease is of fungal origin.

The protease may be a variant of, e.g., a wild-type protease as long as the protease has the thermostability properties defined herein. In one embodiment, the thermostable protease is a variant of a metallo protease as defined above. In one embodiment, the thermostable protease used in a process described herein is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In one embodiment, the thermostable protease is a variant of the mature part of the metallo protease shown in SEQ ID NO: 2 disclosed in WO 2003/048353 or the mature part of SEQ ID NO: 1 in WO 2010/008841 further with one of the following substitutions or combinations of substitutions:

S5*+D79L+S87P+A112P+D142L;

D79L+S87P+A112P+T124V+D142L;

S5*+N26R+D79L+S87P+A112P+D142L;

N26R+T46R+D79L+S87P+A112P+D142L;

T46R+D79L+S87P+T116V+D142L;

D79L+P81R+S87P+A112P+D142L;

A27K+D79L+S87P+A112P+T124V+D142L;

D79L+Y82F+S87P+A112P+T124V+D142L;

D79L+S87P+A112P+T124V+A126V+D142L;

D79L+S87P+A112P+D142L;

D79L+Y82F+S87P+A112P+D142L;

S38T+D79L+S87P+A112P+A126V+D142L;

D79L+Y82F+S87P+A112P+A126V+D142L;

A27K+D79L+S87P+A112P+A126V+D142L;

D79L+S87P+N98C+A112P+G135C+D142L;

D79L+S87P+A112P+D142L+T141C+M1610;

S36P+D79L+S87P+A112P+D142L;

A37P+D79L+S87P+A112P+D142L;

S49P+D79L+S87P+A112P+D142L;

S50P+D79L+S87P+A112P+D142L;

D79L+S87P+D104P+A112P+D142L;

D79L+Y82F+S87G+A112P+D142L;

S70V+D79L+Y82F+S87G+Y97W+A112P+D142L;

D79L+Y82F+S87G+Y97W+D104P+A112P+D142L;

S70V+D79L+Y82F+S87G+A112P+D142L;

D79L+Y82F+S87G+D104P+A112P+D142L;

D79L+Y82F+S87G+A112P+A126V+D142L;

Y82F+S87G+S70V+D79L+D104P+A112P+D142L;

Y82F+S87G+D79L+D104P+A112P+A126V+D142L;

A27K+D79L+Y82F+S87G+D104P+A112P+A126V+D142L;

A27K+Y82F+S87G+D104P+A112P+A126V+D142L;

A27K+D79L+Y82F+D104P+A112P+A126V+D142L;

A27K+Y82F+D104P+A112P+A126V+D142L;

A27K+D79L+S87P+A112P+D142L; and

D79L+S87P+D142L.

In one embodiment, the thermostable protease is a variant of the metallo protease disclosed as the mature part of SEQ ID NO: 2 disclosed in WO 2003/048353 or the mature part of SEQ ID NO: 1 in WO 2010/008841 with one of the following substitutions or combinations of substitutions:

D79L+S87P+A112P+D142L;

D79L+S87P+D142L; and

A27K+D79L+Y82F+S87G+D104P+A112P+A126V+D142L.

In one embodiment, the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841.

The thermostable protease may also be derived from any bacterium as long as the protease has the thermostability properties.

In one embodiment, the thermostable protease is derived from a strain of the archae (previously classified as bacterium) Pyrococcus, such as a strain of Pyrococcus furiosus (pfu protease), for example, the Pyrococcus furiosus protease of SEQ ID NO: 2 or a variant thereof having at least 80% identity, such as at least 85%, such as at least 90%, such as at least 95%, such as at least 96%, such as at least 97%, such as at least 98%, such as at least 99% identity thereto.

In one embodiment, the protease is one shown as SEQ ID NO: 1 in U.S. Pat. No. 6,358,726-B1 (Takara Shuzo Company).

In one embodiment, the thermostable protease is a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1. The Pyroccus furiosus protease can be purchased from Takara Bio, Japan.

The Pyrococcus furiosus protease is a thermostable protease. The commercial product Pyrococcus furiosus protease (PfuS) was found to have a thermostability of 110% (80° C./70° C.) and 103% (90° C./70° C.) at pH 4.5.

In one embodiment, a thermostable protease used in a process described herein has a thermostability value of more than 20% determined as Relative Activity at 80° C./70° C.

In one embodiment, the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In one embodiment, protease has a thermostability of between 20 and 50%, such as between 20 and 40%, such as 20 and 30% determined as Relative Activity at 80° C./70° C. In one embodiment, the protease has a thermostability between 50 and 115%, such as between 50 and 70%, such as between 50 and 60%, such as between 100 and 120%, such as between 105 and 115% determined as Relative Activity at 80° C./70° C.

In one embodiment, the protease has a thermostability value of more than 10% determined as Relative Activity at 85° C./70° C.

In one embodiment, the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In one embodiment, the protease has a thermostability of between 10% and 50%, such as between 10% and 30%, such as between 10% and 25% determined as Relative Activity at 85° C./70° C.

In one embodiment, the protease has more than 20%, more than 30%, more than 40%, more than 50%, more than 60%, more than 70%, more than 80%, more than 90% determined as Remaining Activity at 80° C.; and/or the protease has more than 20%, more than 30%, more than 40%, more than 50%, more than 60%, more than 70%, more than 80%, more than 90% determined as Remaining Activity at 84° C.

Determination of “Relative Activity” and “Remaining Activity” is done as described in the art (e.g., PCT/US2017/063159, filed Nov. 22, 2017).

In one embodiment, the protease may have a themostability for above 90, such as above 100 at 85° C. as determined using a Zein-BCA assay.

In one embodiment, the protease has a themostability above 60%, such as above 90%, such as above 100%, such as above 110% at 85° C. as determined using a Zein-BCA assay.

In one embodiment, protease has a themostability between 60-120, such as between 70-120%, such as between 80-120%, such as between 90-120%, such as between 100-120%, such as 110-120% at 85° C. as determined using a Zein-BCA assay.

In one embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by a AZCL-casein assay.

Additional proteases contemplated for use with the present invention can be found in U.S. Provisional Patent No. 62/514,636 filed Jun. 2, 2017 (the content of which is incorporated herein).

Additional polynucleotides encoding suitable proteases may be obtained from microorganisms of any genus, including those readily available within the UniProtKB database (www.uniprot.org).

The protease coding sequences can also be used to design nucleic acid probes to identify and clone DNA encoding proteases from strains of different genera or species, as described supra.

The polynucleotides encoding proteases may also be identified and obtained from other sources including microorganisms isolated from nature (e.g., soil, composts, water, etc.) or DNA samples obtained directly from natural materials (e.g., soil, composts, water, etc,) as described supra.

Techniques used to isolate or clone polynucleotides encoding proteases are described supra.

The protease can also include fused polypeptides or cleavable fusion polypeptides, as described supra.

In one embodiment, the thermostable protease is a serine protease, e.g., an S8 protease, such as one disclosed in U.S. 62/567,841, filed on Oct. 4, 2017 (Attorney Docket No. 14484-US-PRO), which is hereby incorporated herein by reference in its entirety.

In an embodiment, the S8 protease is derived from Palaeococcus, for instance Palaeococcus ferrophilus, such as the Palaeococcus ferrophilus S8 protease of SEQ ID NO: 9, or a variant thereof having at least 60% identity, preferably at least 65% identity, preferably at least 70% identity, at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, or at least 99%, but less than 100% identity to the amino acid sequence of SEQ ID NO: 9.

In an embodiment, the S8 protease is derived from Thermococcus, for instance Thermococcus litoralis or Thermococcus thioreducens, such as the Thermococcus litoralis S8 protease of SEQ ID NO: 10, or a variant thereof having at least 60% identity, preferably at least 65% identity, preferably at least 70% identity, at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, or at least 99%, but less than 100% identity to the amino acid sequence of SEQ ID NO: 10, or the Thermococcus thioreducens S8 protease of SEQ ID NO: 11, or a variant thereof having at least 60% identity, preferably at least 65% identity, preferably at least 70% identity, at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, or at least 99%, but less than 100% identity to the amino acid sequence of SEQ ID NO: 11.

### Glucoamylase in Liquefaction

A glucoamylase may optionally be present and/or added in liquefaction step step and/or the slurry prior to optional jet cook and/or liquefaction. In one embodiment, the glucoamylase is added together with or separately from the alpha-amylase and/or the optional protease, endoglucanase, phospholipase C, xylanase, phytase, and/or pullulanase.

In some embodiments, the fermenting organism comprises a heterologous polynucleotide encoding a glucoamylase, for example, as described in WO 2017/087330, the content of which is hereby incorporated by reference. Any glucoamylase described or referenced herein is contemplated for expression in the fermenting organism.

The glucoamylase may be any glucoamylase that is suitable for the host cells and/or the methods described herein, such as a naturally occurring glucoamylase or a variant thereof that retains glucoamylase activity.

In one embodiment, the glucoamylase has a Relative Activity heat stability at 85° C. of at least 20%, at least 30%, or at least 35% determined as described in Example 4 of PCT/US2017/063159, filed Nov. 22, 2017 (heat stability).

In one embodiment, the glucoamylase has a relative activity pH optimum at pH 5.0 of at least 90%, e.g., at least 95%, at least 97%, or 100% determined as described in Example 4 of PCT/US2017/063159, filed Nov. 22, 2017 (pH optimum).

In one embodiment, the glucoamylase has a pH stability at pH 5.0 of at least 80%, at least 85%, at least 90% determined as described in Example 4 of PCT/US2017/063159, filed Nov. 22, 2017 (pH stability).

In one embodiment, the glucoamylase, such as a Penicillium oxalicum glucoamylase variant, used in liquefaction has a thermostability determined as DSC Td at pH 4.0 as described in Example 15 of PCT/US2017/063159, filed Nov. 22, 2017 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C. In one embodiment, the glucoamylase, such as a Penicillium oxalicum glucoamylase variant has a thermostability determined as DSC Td at pH 4.0 as described in Example 15 of PCT/US2017/063159, filed Nov. 22, 2017 in the range between 70° C. and 95° C., such as between 80° C. and 90° C.

In one embodiment, the glucoamylase, such as a Penicillium oxalicum glucoamylase variant, used in liquefaction has a thermostability determined as DSC Td at pH 4.8 as described in Example 15 of PCT/US2017/063159, filed Nov. 22, 2017 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C., such as at least 91° C. In one embodiment, the glucoamylase, such as a Penicillium oxalicum glucoamylase variant has a thermostability determined as DSC Td at pH 4.8 as described in Example 15 of PCT/US2017/063159, filed Nov. 22, 2017 in the range between 70° C. and 95° C., such as between 80° C. and 90° C.

In one embodiment, the glucoamylase, such as a Penicillium oxalicum glucoamylase variant, used in liquefaction has a residual activity determined as described in Example 16 of PCT/US2017/063159, filed Nov. 22, 2017, of at least 100% such as at least 105%, such as at least 110%, such as at least 115%, such as at least 120%, such as at least 125%. In one embodiment, the glucoamylase, such as a Penicillium oxalicum glucoamylase variant has a thermostability determined as residual activity as described in Example 16 of PCT/US2017/063159, filed Nov. 22, 2017, in the range between 100% and 130%.

In one embodiment, the glucoamylase, e.g., of fungal origin such as a filamentous fungi, from a strain of the genus Penicillium, e.g., a strain of Penicillium oxalicum, in particular the Penicillium oxalicum glucoamylase disclosed as SEQ ID NO: 2 in WO 2011/127802 (which is hereby incorporated by reference) and shown in SEQ ID NO: 12.

In one embodiment, the glucoamylase has at least 80%, e.g., at least 85%, at least 90%, at least 91%, at least 92%, at least 93%, at least 94%, at least 95%, at least 96%, at least 97%, at least 98%, at least 99% or 100% identity to the mature polypeptide shown in SEQ ID NO: 2 in WO 2011/127802 or SEQ ID NO: 12 herein.

In one embodiment, the glucoamylase is a variant of the Penicillium oxalicum glucoamylase disclosed as SEQ ID NO: 2 in WO 2011/127802 and shown in SEQ ID NO: 12, having a K79V substitution. The K79V glucoamylase variant has reduced sensitivity to protease degradation relative to the parent as disclosed in WO 2013/036526 (which is hereby incorporated by reference).

In one embodiment, the glucoamylase is derived from Penicillium oxalicum.

In one embodiment, the glucoamylase is a variant of the Penicillium oxalicum glucoamylase disclosed as SEQ ID NO: 2 in WO 2011/127802. In one embodiment, the Penicillium oxalicum glucoamylase is the one disclosed as SEQ ID NO: 2 in WO 2011/127802 having Val (V) in position 79.

Contemplated Penicillium oxalicum glucoamylase variants are disclosed in WO 2013/053801 which is hereby incorporated by reference.

In one embodiment, these variants have reduced sensitivity to protease degradation.

In one embodiment, these variant have improved thermostability compared to the parent.

In one embodiment, the glucoamylase has a K79V substitution (using SEQ ID NO: 2 of WO 2011/127802 for numbering), corresponding to the PE001 variant, and further comprises one of the following alterations or combinations of alterations

T65A; Q327F; E501V; Y504T; Y504*; T65A+Q327F; T65A+E501V; T65A+Y504T; T65A+Y504*; Q327F+E501V; Q327F+Y504T; Q327F+Y504*; E501V+Y504T; E501V+Y504*; T65A+Q327F+E501V; T65A+Q327F+Y504T; T65A+E501V+Y504T; Q327F+E501V+Y504T; T65A+Q327F+Y504*; T65A+E501V+Y504*; Q327F+E501V+Y504*; T65A+Q327F+E501V+Y504T; T65A+Q327F+E501V+Y504*; E501V+Y504T; T65A+K161S; T65A+Q405T; T65A+Q327W; T65A+Q327F; T65A+Q327Y; P11F+T65A+Q327F; R1K+D3W+K5Q+G7V+N8S+T10K+P11S+T65A+Q327F; P2N+P4S+P11F+T65A+Q327F; P11F+D26C+K33C+T65A+Q327F; P2N+P4S+P11F+T65A+Q327W+E501V+Y504T; R1E+D3N+P4G+G6R+G7A+N8A+T10D+P11D+T65A+Q327F; P11F+T65A+Q327W; P2N+P4S+P11F+T65A+Q327F+E501V+Y504T; P11F+T65A+Q327W+E501V+Y504T; T65A+Q327F+E501V+Y504T; T65A+S105P+Q327W; T65A+S105P+Q327F; T65A+Q327W+S364P; T65A+Q327F+S364P; T65A+S103N+Q327F; P2N+P4S+P11F+K34Y+T65A+Q327F; P2N+P4S+P11F+T65A+Q327F+D445N+V447S; P2N+P4S+P11F+T65A+I172V+Q327F; P2N+P4S+P11F+T65A+Q327F+N502*; P2N+P4S+P11F+T65A+Q327F+N502T+P563S+K571E; P2N+P4S+P11F+R31S+K33V+T65A+Q327F+N564D+K571S; P2N+P4S+P11F+T65A+Q327F+S377T; P2N+P4S+P11F+T65A+V325T+Q327W; P2N+P4S+P11F+T65A+Q327F+D445N+V447S+E501V+Y504T; P2N+P4S+P11F+T65A+I172V+Q327F+E501V+Y504T; P2N+P4S+P11F+T65A+Q327F+S377T+E501V+Y504T; P2N+P4S+P11F+D26N+K34Y+T65A+Q327F; P2N+P4S+P11F+T65A+Q327F+I375A+E501V+Y504T; P2N+P4S+P11F+T65A+K218A+K221D+Q327F+E501V+Y504T; P2N+P4S+P11F+T65A+S103N+Q327F+E501V+Y504T; P2N+P4S+T10D+T65A+Q327F+E501V+Y504T; P2N+P4S+F12Y+T65A+Q327F+E501V+Y504T; K5A+P11F+T65A+Q327F+E501V+Y504T; P2N+P4S+T10E+E18N+T65A+Q327F+E501V+Y504T; P2N+T10E+E18N+T65A+Q327F+E501V+Y504T; P2N+P4S+P11F+T65A+Q327F+E501V+Y504T+T568N; P2N+P4S+P11F+T65A+Q327F+E501V+Y504T+K524T+G526A; P2N+P4S+P11F+K34Y+T65A+Q327F+D445N+V447S+E501V+Y504T; P2N+P4S+P11F+R31S+K33V+T65A+Q327F+D445N+V447S+E501V+Y504T; P2N+P4S+P11F+D26N+K34Y+T65A+Q327F+E501V+Y504T; P2N+P4S+P11F+T65A+F80*+Q327F+E501V+Y504T; P2N+P4S+P11F+T65A+K112S+Q327F+E501V+Y504T; P2N+P4S+P11F+T65A+Q327F+E501V+Y504T+T516P+K524T+G526A; P2N+P4S+P11F+T65A+Q327F+E501V+N502T+Y504*; P2N+P4S+P11F+T65A+Q327F+E501V+Y504T; P2N+P4S+P11F+T65A+S103N+Q327F+E501V+Y504T; K5A+P11F+T65A+Q327F+E501V+Y504T; P2N+P4S+P11F+T65A+Q327F+E501V+Y504T+T516P+K524T+G526A; P2N+P4S+P11F+T65A+V79A+Q327F+E501V+Y504T; P2N+P4S+P11F+T65A+V79G+Q327F+E501V+Y504T; P2N+P4S+P11F+T65A+V791+Q327F+E501V+Y504T; P2N+P4S+P11F+T65A+V79L+Q327F+E501V+Y504T; P2N+P4S+P11F+T65A+V79S+Q327F+E501V+Y504T; P2N+P4S+P11F+T65A+L72V+Q327F+E501V+Y504T; S255N+Q327F+E501V+Y504T; P2N+P4S+P11F+T65A+E74N+V79K+Q327F+E501V+Y504T; P2N+P4S+P11F+T65A+G220N+Q327F+E501V+Y504T; P2N+P4S+P11F+T65A+Y245N+Q327F+E501V+Y504T; P2N+P4S+P11F+T65A+Q253N+Q327F+E501V+Y504T; P2N+P4S+P11F+T65A+D279N+Q327F+E501V+Y504T; P2N+P4S+P11F+T65A+Q327F+S359N+E501V+Y504T; P2N+P4S+P11F+T65A+Q327F+D370N+E501V+Y504T; P2N+P4S+P11F+T65A+Q327F+V460S+E501V+Y504T; P2N+P4S+P11F+T65A+Q327F+V460T+P468T+E501V+Y504T; P2N+P4S+P11F+T65A+Q327F+T463N+E501V+Y504T; P2N+P4S+P11F+T65A+Q327F+S465N+E501V+Y504T; and P2N+P4S+P11F+T65A+Q327F+T477N+E501V+Y504T.

In one embodiment, the Penicillium oxalicum glucoamylase variant has a K79V substitution (using SEQ ID NO: 2 of WO 2011/127802 for numbering), corresponding to the PE001 variant, and further comprises one of the following substitutions or combinations of substitutions:

P11F+T65A+Q327F;

P2N+P4S+P11F+T65A+Q327F;

P11F+D26C+K33C+T65A+Q327F;

P2N+P4S+P11F+T65A+Q327W+E501V+Y504T;

P2N+P4S+P11F+T65A+Q327F+E501V+Y504T; and

P11F+T65A+Q327W+E501V+Y504T.

The glucoamylase may be added in amounts from 0.1-100 micrograms EP/g, such as 0.5-50 micrograms EP/g, such as 1-25 micrograms EP/g, such as 2-12 micrograms EP/g DS.

In one embodiment, the glucoamylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any glucoamylase described or referenced herein. In one aspect, the glucoamylase sequence differs by no more than ten amino acids, e.g., by no more than five amino acids, by no more than four amino acids, by no more than three amino acids, by no more than two amino acids, or by one amino acid from any glucoamylase described or referenced herein. In one embodiment, the glucoamylase comprises or consists of the amino acid sequence of any glucoamylase described or referenced herein, allelic variant, or a fragment thereof having glucoamylase activity. In one embodiment, the glucoamylase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the glucoamylase has at least 20%, e.g., at least 40%, at least 50%, at least 60%, at least 70%, at least 80%, at least 90%, at least 95%, at least 96%, at least 97%, at least 98%, at least 99%, or 100% of the glucoamylase activity of any glucoamylase described or referenced herein under the same conditions.

In some embodiments, the glucoamylase comprises a variant of Penicillium oxalicum glucoamylase having the following mutations: K79V+P2N+P4S+P11F+T65A+Q327F (using SEQ ID NO: 11 herein for numbering).

### Phospholipase C in Liquefaction

A phospholipase C may optionally be present and/or added in liquefaction step and/or the slurry prior to optional jet cook and/or liquefaction. In one embodiment, the phospholipase C is added together with or separately from the alpha-amylase, triacylglycerol lipase, and/or the optional protease, endoglucanase, phospholipase C, xylanase, phytase, and/or pullulanase.

Examples of suitable phospholipase C polypeptides are described in WO2017/112542, which is incorporated herein by reference in its entirety. In one embodiment, the phospholipase C is a Penicillium emersonii PLC (PePLC) having the amino acid sequence of SEQ ID NO: 2 therein, or a variant thereof having at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 2 therein. In one embodiment, the phospholipase C is a Trichoderma harzianum PLC having the amino acid sequence of SEQ ID NO: 7 therein or SEQ ID NO: 8 therein, or a variant thereof having at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 7 therein or SEQ ID NO: 8 therein. In an embodiment, the lipase added to liquefaction, saccharification, fermentation, and/or simultaneous saccharification and fermentation is not a phospholipase C. In an embodiment, liquefaction, saccharification, fermentation, and/or simultaneous saccharification and fermentation is carried out in the absence of a phospholipase C, optionally in the absence of a PePLC, or a Trichoderma harzianum phospholipase C.

### B. Saccharification Step (b)

Liquefaction step (a) is followed by saccharification of dextrins in step (b). According to the present disclosure a triacylgylceride lipase is added before or during saccharification step (b). The triacylglyceride lipase can be added before or during saccharification step (b) independently of addition of a triacylgylceride lipase to liquefaction step (a), or subsequent to addition of the triacylglyceride lipase to liquefaction step (a)

In an embodiment a process of the disclosure may comprise a pre-saccharification step, i.e., after step (a), but before saccharification step (b), carried out for 40-90 minutes at a temperature between 30-65° C. According to the present disclosure a triacylgylceride lipase is added during the pre-saccharification step between liquefaction step (a) and saccharification step (b). The triacylglyceride lipase can be added during the pre-saccharification step independently of addition of a triacylgylceride lipase to liquefaction step (a) or saccharification step (b), or subsequent to addition of the triacylglyceride lipase to liquefaction step (a) and before subsequent addition of a triacylglyceride lipase to saccharification step (b).

According to the present disclosure saccharification step (b) may be carried out at a temperature from 20-75° C., preferably from 40-70° C., such as around 60° C., and at a pH between 4 and 5.

In a preferred embodiment fermentation step (c) or simultaneous saccharification and fermentation (SSF) (i.e., combined steps (b) and (c)) may be carried out at a temperature between 20-60° C., preferably between 25-40° C., such as around 32° C. In an embodiment fermentation step (c) or simultaneous saccharification and fermentation (SSF) are ongoing for 6 to 120 hours, in particular 24 to 96 hours.

According to the present disclosure a triacylglyceride lipase, preferably a thermostable triacylglyceride lipase (e.g., one having a melting point (DSC) of at least 65C), is present and/or added during saccharification step (b) and/or fermentation step (c) or simultaneous saccharification step (b) and fermentation step (c) (SSF). The triacylglyceride lipase added in this manner may be in addition to a triacylglyericide lipase added during liquefaction step (a) and/or during a pre-saccharificaiton step between step (a) and (b) and/or (c).

According to the present disclosure a carbohydrate-source generating enzyme, preferably a glucoamylase, is present and/or added during saccharification step (b) and/or fermentation step (c) or simultaneous saccharification step (b) and fermentation step (c) (SSF).

The term “carbohydrate-source generating enzyme” includes any enzymes generating fermentable sugars. A carbohydrate-source generating enzyme is capable of producing one or more carbohydrates that can be used as an energy source by the fermenting organism(s) in question, for instance, when used in a process of the disclosure for producing ethanol. The generated carbohydrates may be converted directly or indirectly to the desired fermentation product, preferably ethanol. According to the disclosure a mixture of carbohydrate-source generating enzymes may be used.

Specific examples of carbohydrate-source generating enzyme activities include glucoamylase (being glucose generators), beta-amylase and maltogenic amylase (being maltose generators). A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference. The maltogenic amylase may in a preferred embodiment be added in an amount of 0.05-5 mg total protein/gram DS or 0.05-5 MANU/g DS.

In a preferred embodiment the carbohydrate-source generating enzyme is a glucoamylase.

The process of the disclosure, including steps (b) and/or (c), may be carried out using any suitable glucoamylase. The glucoamylase may be of any origin, in particular of fungal origin.

Contemplated glucoamylases include those from the group consisting of Aspergillus glucoamylases, in particular A. nigerG1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, A. oryzae glucoamylase (AgriC. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1 199-1204.

Other glucoamylases contemplated include glucoamylase derived from a strain of Athelia, preferably a strain of Athelia rolfsii (previously denoted Corticium rolfsii) glucoamylase (see U.S. Pat. No. 4,727,026 and (Nagasaka, Y. et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (US patent no. Re. 32,153), Talaromyces duponti, Talaromyces thermophilus (U.S. Pat. No. 4,587,215). Also contemplated are Trichoderma reesei glucoamylases including the one disclosed as SEQ ID NO: 4 in WO 2006/060062 and glucoamylases being at least 80% or at least 90% identical thereto (hereby incorporated by reference).

In an embodiment the glucoamylase is derived from a strain of Aspergillus, preferably A. niger, A. awamori, or A. oryzae; or a strain of Trichoderma, preferably T. reesei; or a strain of Talaromyces, preferably T. emersonii.

In an embodiment the glucoamylase present and/or added during saccharification step (b) and/or fermentation step (c) is of fungal origin, such as from a strain of Pycnoporus, or a strain of Gloephyllum. In an embodiment the glucoamylase is derived from a strain of the genus Pycnoporus, in particular a strain of Pycnoporus sanguineus described in WO 2011/066576 (SEQ ID NOs 2, 4 or 6), such as the one shown as SEQ ID NO: 4 in WO 2011/066576.

In a preferred embodiment the glucoamylase is derived from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is the Gloeophyllum sepiarium shown in SEQ ID NO: 2 in WO 2011/068803.

Other contemplated glucoamylases include glucoamylase derived from a strain of Trametes, preferably a strain of Trametes cingulata disclosed as SEQ ID NO: 34 in WO 2006/069289 (which is hereby incorporated by reference).

Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; SANT® SUPER, SANT™ EXTRA L, SPIRIZYME™ PLUS, SPIRIZYME™ FUEL, SPIRIZYME™ ULTRA, SPIRIZYME™ EXCEL, SPIRIZYME™ ACHIEVE, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.05-5 AGU/g DS (in whole stillage), especially between 0.1-2 AGU/g DS.

Glucoamylase may be added in an effective amount, preferably in the range from 0.001-1 mg enzyme protein per g DS, preferably 0.01-0.5 mg enzyme protein per g dry solid (DS).

Optionally an alpha-amylase (EC 3.2.1.1) may be added during saccharification step (b) and/or fermentation step (c). The alpha-amylase may be of any origin, but is typically of filamentous fungus origin. According to the disclosure an alpha-amylases adding during saccharification and/or fermentation is typically a fungal acid alpha-amylase.

The fungal acid alpha-amylases may be an acid fungal alpha-amylase derived from a strain of the genus Aspergillus, such as Aspergillus oryzae and Aspergillus niger.

A suitable fungal acid alpha-amylase is one derived from a strain Aspergillus niger. In a preferred embodiment the fungal acid alpha-amylase is the one from A. niger disclosed as “AMYA_ASPNG” in the Swiss-prot/TeEMBL database under the primary accession no. P56271 and described in more detail in WO 89/01969 (Example 3). The acid Aspergillus niger acid alpha-amylase is also shown as SEQ ID NO: 1 in WO 2004/080923 (Novozymes) which is hereby incorporated by reference. Also variants of said acid fungal amylase having at least 70% identity, such as at least 80% or even at least 90% identity, such as at least 95%, at least 96%, at least 97%, at least 98%, or at least 99% identity to SEQ ID NO: 1 in WO 2004/080923 are contemplated. A suitable commercially available acid fungal alpha-amylase derived from Aspergillus niger is SP288 (available from Novozymes A/S, Denmark).

The fungal acid alpha-amylase may also be a wild-type enzyme comprising a carbohydrate-binding module (CBM) and an alpha-amylase catalytic domain (i.e., a non-hybrid), or a variant thereof. In an embodiment the wild-type fungal acid alpha-amylase is derived from a strain of Aspergillus kawachii.

A specific example of a contemplated hybrid alpha-amylase includes the Rhizomucor pusillus alpha-amylase with Aspergillus niger glucoamylase linker and starch-binding domain (SBD) (which is disclosed in Table 5 as a combination of amino acid sequences SEQ ID NO: 20, SEQ ID NO: 72 and SEQ ID NO: 96 in U.S. application Ser. No. 11/316,535) (hereby incorporated by reference). In another embodiment the hybrid fungal acid alpha-amylase is a Meripilus giganteus alpha-amylase with Athelia rolfsii glucoamylase linker and SBD (SEQ ID NO: 102 in U.S. 60/638,614) (hereby incorporated by reference). Other specific examples of contemplated hybrid alpha-amylases include those disclosed in U.S. Patent Publication no. 2005/0054071, including those disclosed in Table 3 on page 15, such as Aspergillus niger alpha-amylase with Aspergillus kawachii linker and starch binding domain.

In a preferred embodiment the fungal acid alpha-amylase is one disclosed in WO 2013/006756 including the following variants: Rhizomucor pusillus alpha-amylase variant having an Aspergillus niger glucoamylase linker and starch-binding domain (SBD) which further comprises at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C.

An acid alpha-amylase may according to the present disclosure be added in an amount of 0.1 to 10 AFAU/g DS, preferably 0.10 to 5 AFAU/g DS, especially 0.3 to 2 AFAU/g DS.

### C. Fermenting Organisms

The term “fermenting organism” refers to any organism, including bacterial and fungal organisms, especially yeast, suitable for use in a fermentation process and capable of producing the desired fermentation product.

Examples of fermenting organisms used in fermentation step (c) or simultaneous saccharification and fermentation (i.e., SSF) for converting fermentable sugars in the fermentation medium into fermentation products, such as especially ethanol, include fungal organisms, such as especially yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

“Fermentation medium” refers to the environment in which fermentation is carried out. The fermentation medium includes the fermentation substrate, that is, the carbohydrate source that is metabolized by the fermenting organism. According to the present disclosure the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

Examples of commercially available yeast includes, e.g., RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### D. Starch-Containing Materials

Any suitable starch-containing material may be used as starting material according to the present disclosure. Examples of starch-containing materials, suitable for use in a process of the disclosure, include whole grains, corn, wheat, barley, rye, milo, sago, cassava, tapioca, sorghum, rice, peas, beans, or sweet potatoes, or mixtures thereof or starches derived there from, or cereals. Contemplated are also waxy and non-waxy types of corn and barley.

In a preferred embodiment the starch-containing material, used for fermentation product production, such as especially ethanol production, is corn or wheat.

### E. Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the present disclosure are used for producing an alcohol, such as ethanol. The fermentation product, such as ethanol, obtained according to the present disclosure, may be used as fuel, which is typically blended with gasoline. However, in the case of ethanol it may also be used as potable ethanol.

### F. Recovery

Subsequent to fermentation, or SSF, the fermentation product may be separated from the fermentation medium. The slurry may be distilled to extract the desired fermentation product (e.g., ethanol). Alternatively the desired fermentation product may be extracted from the fermentation medium by micro or membrane filtration techniques. The fermentation product may also be recovered by stripping or other method well known in the art.

### III. Uses

In yet another aspect, the present disclosure relates to the use of a triacylgycerol lipase during liquefaction, pre-saccharification, saccharification, fermentation, and/or simultaneous saccharification and fermentation in a fermentation product production process for increasing enzymatically accessible starch and/or yield of a fermentation product (e.g., ethanol yield).

Any triacylgycerol lipase, for example a triacylgycerol lipase described above, can be used in a liquefaction step, pre-saccharification, saccharification, fermentation, and/or simultaneous saccharification and fermentation of an ethanol production process to increase enzymatically accessible starch and/or ethanol yield.

In preferred embodiments, the triacylgycerol lipase is of fungal origin (e.g, a thermostable fungal triacylglycerol lipase).

In preferred embodiments, the triacylgycerol lipase used in the liquefaction step, pre-saccharification step, saccharification step, fermentation step, and/or simultaneous saccharification or fermentation step has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3 herein, preferably derived from a strain of the genus Rhizomucor, such as a strain of Rhizomucor miehei.

In preferred embodiments, the triacylgycerol lipase used in the liquefaction step, pre-saccharification step, saccharification step, fermentation step, and/or simultaneous saccharification or fermentation step has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the polypeptide of SEQ ID NO: 4 herein, preferably derived from a strain of the genus Aspergillus, such as a strain of Aspergillus oryzae.

In preferred embodiments, the triacylgycerol lipase used in the liquefaction step, pre-saccharification step, saccharification step, fermentation step, and/or simultaneous saccharification or fermentation step has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO:5 herein or SEQ ID NO: 6 herein, preferably derived from a strain of the genus Moesziomyces, such as a strain of Moesziomyces antarcticus.

In preferred embodiments, the triacylgycerol lipase used in the liquefaction step, pre-saccharification step, saccharification step, fermentation step, and/or simultaneous saccharification or fermentation step has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO:7 herein or SEQ ID NO: 8 herein, preferably derived from a strain of the genus Thermomyces, such as a strain of Thermomyces lanuginosus.

### IV. Examples of Preferred Embodiments of the Disclosure

In a preferred embodiment the present disclosure relates to a process for producing ethanol from starch-containing material comprising the steps of: (a) liquefying the starch-containing material at a pH in the range between 4.0-6.5 at a temperature in the range from 70-100° C. using: —an alpha-amylase derived from Bacillus stearothermophilus; —a triacylgycerol lipase, preferably having a Melting Point (DSC) of at least about 65° C.; (b) saccharifying using a glucoamylase enzyme; and (c) fermenting using a fermenting organism. In an embodiment, a triacylgycerol lipase is also added before or during saccharifying step (b), fermenting step (c) or simultaneous saccharification and fermentation. In an embodiment, a triacylgycerol lipase is also added during a pre-saccharification step between steps (a) and (b).

In a preferred embodiment the present disclosure relates to a process for producing ethanol from starch-containing material comprising the steps of: (a) liquefying the starch-containing material at a pH in the range between 4.0-6.5 at a temperature in the range from 70-100° C. using an alpha-amylase derived from Bacillus stearothermophilus; (b) saccharifying using a glucoamylase enzyme; and (c) fermenting using a fermenting organism, wherein a triacylgycerol lipase, preferably having a Melting Point (DSC) of at least about 65° C. is added before or during saccharifying step (b), fermenting step (c) or simultaneous saccharification and fermentation. In an embodiment, a triacylgycerol lipase is also added before or during liquefying step (b). In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.5-6.2 at a temperature above the initial gelatinization temperature using:—an alpha-amylase, preferably derived from Bacillus stearothermophilus, having a T½ (min) at pH 4.5, 85° C., 0.12 mM CaCl₂ of at least 10; —a triacylgycerol lipase, preferably having a Melting Point (DSC) of at least about 65° C.; (b) saccharifying using a glucoamylase enzyme; and (c) fermenting using a fermenting organism. In an embodiment, a triacylgycerol lipase is also added before or during saccharifying step (b), fermenting step (c) or simultaneous saccharification and fermentation. In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.5-6.2 at a temperature above the initial gelatinization temperature using an alpha-amylase, preferably derived from Bacillus stearothermophilus, having a T½ (min) at pH 4.5, 85° C., 0.12 mM CaCl₂) of at least 10; (b) saccharifying using a glucoamylase enzyme; and (c) fermenting using a fermenting organism, wherein a triacylgycerol lipase, preferably having a Melting Point (DSC) of at least about 65° C. is added before or during saccharifying step (b), fermenting step (c) or simultaneous saccharification and fermentation. In an embodiment, a triacylgycerol lipase is also added before or during liquefying step (b). In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.0-6.5 at a temperature between 70-100° C. using: —a bacterial alpha-amylase, preferably derived from Bacillus stearothermophilus, having a T½ (min) at pH 4.5, 85° C., 0.12 mM CaCl₂ of at least 10; —a triacylgycerol lipase, preferably having a Melting Point (DSC) of at least about 65° C.; (b) saccharifying using a glucoamylase enzyme; and (c) fermenting using a fermenting organism.

In an embodiment, a triacylgycerol lipase is also added before or during saccharifying step (b), fermenting step (c) or simultaneous saccharification and fermentation. In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.0-6.5 at a temperature between 70-100° C. using a bacterial alpha-amylase, preferably derived from Bacillus stearothermophilus, having a T½ (min) at pH 4.5, 85° C., 0.12 mM CaCl₂ of at least 10; (b) saccharifying using a glucoamylase enzyme; and (c) fermenting using a fermenting organism, wherein a triacylgycerol lipase, preferably having a Melting Point (DSC) of at least about 65° C. is added before or during saccharifying step (b), fermenting step (c) or simultaneous saccharification and fermentation. In an embodiment, a triacylgycerol lipase is also added before or during liquefying step (b). In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.0-6.5 at a temperature above the initial gelatinization temperature using: —an alpha-amylase shown in SEQ ID NO: 1 having a double deletion in positions R179+G180 or I181+G182, and optional substitution N193F; and optionally further one of the following set of substitutions:—E129V+K177L+R179E; —V59A+Q89R+E129V+K177L+R179E+H208Y+K220P+N224L+Q254S; —E129V+K177L+R179E+K220P+N224L+S242Q+Q254S; —V59A+Q89R+E129V+K177L+R179E+Q254S+M284V (using SEQ ID NO: 1 herein for numbering); —a triacylgycerol lipase, preferably having a Melting Point (DSC) of at least about 80° C., such as a triacylgycerol lipase having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3, SEQ ID NO: 4, SEQ ID NO: 5, SEQ ID NO: 6, SEQ ID NO: 7, or SEQ ID NO: 8 herein; (b) saccharifying using a glucoamylase enzyme; (c) fermenting using a fermenting organism. In an embodiment, a triacylgycerol lipase is also added before or during saccharifying step (b), fermenting step (c) or simultaneous saccharification and fermentation. In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.0-6.5 at a temperature above the initial gelatinization temperature using an alpha-amylase shown in SEQ ID NO: 1 having a double deletion in positions R179+G180 or I181+G182, and optional substitution N193F; and optionally further one of the following set of substitutions:—E129V+K177L+R179E; —V59A+Q89R+E129V+K177L+R179E+H208Y+K220P+N224L+Q254S; —E129V+K177L+R179E+K220P+N224L+S242Q+Q254S; —V59A+Q89R+E129V+K177L+R179E+Q254S+M284V (using SEQ ID NO: 1 herein for numbering); (b) saccharifying using a glucoamylase enzyme; (c) fermenting using a fermenting organism, wherein a triacylgycerol lipase, preferably having a Melting Point (DSC) of at least about 80° C., such as a triacylgycerol lipase having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3, SEQ ID NO: 4, SEQ ID NO: 5, SEQ ID NO: 6, SEQ ID NO: 7, or SEQ ID NO: 8 herein, is added before or during saccharifying step (b), fermenting step (c) or simultaneous saccharification and fermentation. In an embodiment, a triacylgycerol lipase is also added before or during liquefying step (b). In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.0-6.5 at a temperature between 70-100° C. using: —an alpha-amylase derived from Bacillus stearothermophilus having a double deletion in positions I181+G182, and optional substitution N193F; and optionally further one of the following set of substitutions: —V59A+Q89R+E129V+K177L+R179E+H208Y+K220P+N224L+Q254S; or —V59A+Q89R+E129V+K177L+R179E+Q254S+M284V (using SEQ ID NO: 1 herein for numbering); —a triacylgycerol lipase, preferably having a Melting Point (DSC) of at least about 65° C., about 73° C., about 86° C., or about 90° C.; such as a triacylgycerol lipase having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3, SEQ ID NO: 4, SEQ ID NO: 5, SEQ ID NO: 6, SEQ ID NO: 7, or SEQ ID NO: 8 herein; and—optionally a Penicillium oxalicum glucoamylase in SEQ ID NO:12 herein, preferably having substitutions selected from the group of: —K79V; or —K79V+P11F+T65A+Q327F; or —K79V+P2N+P4S+P11F+T65A+Q327F (using SEQ ID NO: 12 herein for numbering); (b) saccharifying using a glucoamylase enzyme; (c) fermenting using a fermenting organism. In an embodiment, a triacylgycerol lipase is also added before or during saccharifying step (b), fermenting step (c) or simultaneous saccharification and fermentation. In an embodiment, a triacylgycerol lipase is also added during a pre-saccharification step between steps (a) and (b).

In a preferred embodiment the process of the disclosure comprises the steps of: (a) liquefying the starch-containing material at a pH in the range between 4.0-6.5 at a temperature between 70-100° C. using:—an alpha-amylase derived from Bacillus stearothermophilus having a double deletion in positions I181+G182, and optional substitution N193F; and optionally further one of the following set of substitutions: —V59A+Q89R+E129V+K177L+R179E+H208Y+K220P+N224L+Q254S; or —V59A+Q89R+E129V+K177L+R179E+Q254S+M284V (using SEQ ID NO: 1 herein for numbering); and optionally a Penicillium oxalicum glucoamylase in SEQ ID NO: 12 herein, preferably having substitutions selected from the group of:—K79V; or —K79V+P11F+T65A+Q327F; or—K79V+P2N+P4S+P11F+T65A+Q327F (using SEQ ID NO: 12 herein for numbering); (b) saccharifying using a glucoamylase enzyme; (c) fermenting using a fermenting organism, wherein a triacylgycerol lipase, preferably having a Melting Point (DSC) of at least about 65° C., about 73° C., about 86° C., or about 90° C.; such as a triacylgycerol lipase having at least 60%, such as at least 70%, such as at least 75% identity, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3, SEQ ID NO: 4, SEQ ID NO: 5, SEQ ID NO: 6, SEQ ID NO: 7, or SEQ ID NO: 8 herein, is added before or during saccharifying step (b), fermenting step (c) or simultaneous saccharification and fermentation. In an embodiment, a triacylgycerol lipase is also added before or during liquefying step (b). In an embodiment, a triacylgycerol lipase is also added during a pre-saccharification step between steps (a) and (b).

In another preferred embodiment the disclosure relates to processes of producing ethanol, comprising: (a) liquifying a starch-containing material using the alpha-amylase shown as SEQ ID NO: 1 or an alpha-amylase having at least 60%, at least 70%, at least 80%, at least 90%, at least 95%, at least 97%, at least 99% sequence identity to SEQ ID NO: 1; (b) saccharifying the liquified starch-containing material using a carbohydrate-source generating enzyme, in particular a glucoamylase, to form fermentable sugars; (c) fermenting the fermentable sugars into ethanol using a fermenting organism; wherein the triacylglyceride lipase shown as SEQ ID NO: 3, SEQ ID NO: 4, SEQ ID NO: 5, SEQ ID NO: 6, SEQ ID NO: 7, or SEQ ID NO: 8, or a triacylglyceride lipase having at least 60%, at least 70%, at least 80%, at least 90%, at least 95%, at least 97%, at least 99% sequence identity to SEQ ID NO: 3, SEQ ID NO: 4, SEQ ID NO: 5, SEQ ID NO: 6, SEQ ID NO: 7, or SEQ ID NO: 8 is added before or during step (a), after step (a) and before step (b), before or during step (b), before or during step (c), or before or during simultaneous step (b) and (c).

In a preferred embodiment a cellulase or cellulolytic enzyme composition is present and/or added during fermentation or simultaneous saccharification and fermentation.

In a preferred embodiment a cellulase or cellulolytic enzyme composition derived from Trichoderma reesei is present and/or added during fermentation or simultaneous saccharification and fermentation (SSF).

In a preferred embodiment a cellulase or cellulolytic enzyme composition and a glucoamylase are present and/or added during fermentation or simultaneous saccharification and fermentation.

In an embodiment the cellulase or cellulolytic enzyme composition is derived from Trichoderma reesei, Humicola insolens, Chrysosporium lucknowense or Penicillium decumbens.

The invention is further summarized in the following paragraphs:

1. A process for increasing enzymatically accessible starch, for example by reducing starch retrogradation, and/or increasing fermentation product yield, such as especially ethanol, during a fermentation product production process, wherein a triacylglycerol lipase is present and/or added before or during a liquefaction step and/or before or during a saccharification step, a fermentation step, or a simultaneous saccharification and fermentation step of the fermentation product production process.

2. A process for producing a fermentation product, comprising the steps of:

(a) liquefying a starch-containing material using an alpha-amylase;
(b) saccharifying the liquefied starch-containing material using a carbohydrate-source generating enzyme to form fermentable sugars; and
(c) fermenting the fermentable sugars using a fermenting organism to product the fermentation product, wherein a triacylglycerol lipase is added before or during liquefying step (a) and/or added before or during saccharifying step (b), fermenting step (c) or simultaneous saccharification and fermentation.

3. The process of paragraph 1 or 2, wherein the triacylglycerol lipase is a thermostable triacylglycerol lipase, preferably having a Melting Point (DSC) of greater than or equal to about 60° C., such as between 60° C. and 110° C., such as between 65° C. and 95° C., such as between 70° C. and 90° C., such as above 70° C., such as above 72° C., such as above 80° C., such as above 85° C., such as above 90° C., such as above 92° C., such as above 94° C., such as above 96° C., such as above 98° C., such as above 100° C.

4. The process of any of paragraphs 1-3, wherein the triacylglycerol lipase has: (i) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 96 percent, at least 97 percent, at least 98 percent, at least 99 percent, such as 100 percent identity to the mature part of the polypeptide of SEQ ID NO: 3 herein, preferably derived from a strain of the genus Rhizomucor, such as a strain of Rhizomucor miehei; (ii) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 96 percent, at least 97 percent, at least 98 percent, at least 99 percent, such as 100 percent identity to the mature part of the polypeptide of SEQ ID NO: 4 herein, preferably derived from a strain of the genus Aspergillus, such as a strain of Aspergillus oryzae; (iii) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 98 percent, at least 97 percent, at least 98 percent, at least 99 percent, such as 100 percent identity to the mature part of the polypeptide of SEQ ID NO: 5 or SEQ ID NO: 6 herein, preferably derived from a strain of the genus Moesziomyces, such as a strain of Moesziomyces antarcticus; or (iii) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 98 percent, at least 97 percent, at least 98 percent, at least 99 percent, such as 100 percent identity to the mature part of the polypeptide of SEQ ID NO: 7 or SEQ ID NO: 8 herein, preferably derived from a strain of the genus Thermomyces, such as a strain of Thermomyces lanuginosus.

5. The process of any of paragraphs 1-4, wherein an alpha-amylase and a triacylglyceride lipase having a Melting Point (DSC) above 72 degrees centigrade, preferably above 80 degrees centigrade, preferably above 82 degrees centigrade, especially at least 86 degrees centigrade, in particular 90 degrees centigrade, are present and/or added in liquefaction step (a), saccharification step (b), simultaneous saccharification and fermentation, or after step (a) and before step (b) during pre-saccharification.

6. The process of any of paragraphs 1-5, further comprises, prior to the liquefaction step a), the steps of:

i) reducing the particle size of the starch-containing material, preferably by dry milling; and
ii) forming a slurry comprising the starch-containing material and water.

7. The process of any of paragraphs 1-6, wherein the pH during liquefaction is between 4.0-6.5, such as 4.5-6.2, such as above 4.8-6.0, such as between 5.0-5.8.

8. The process of any of paragraphs 1-7, wherein the temperature during liquefaction is in the range from 70-100 degrees centigrade, such as between 70-95 degrees centigrade, such as between 75-90 degrees centigrade, preferably between 80-90 degrees centigrade, such as around 85 degrees centigrade.

9. The process of any of paragraphs 1-8, wherein a jet-cooking step is carried out before liquefaction in step a).

10. The process of any of paragraphs 1-9, wherein saccharification and fermentation is carried out sequentially or simultaneously.

11. The process of any of paragraphs 1-10, wherein saccharification is carried out at a temperature from 20-75 degrees centigrade, preferably from 40-70 degrees centigrade, such as around 80 degrees centigrade, and at a pH between 4 and 5.

12. The process of any of paragraphs 1-11, wherein fermentation or simultaneous saccharification and fermentation (SSF) is carried out at a temperature from 25 degrees centigrade to 40 degrees centigrade, such as from 28 degrees centigrade to 35 degrees centigrade, such as from 30 degrees centigrade to 34 degrees centigrade, preferably around about 32 degrees centigrade, such as for 6 to 120 hours, in particular 24 to 98 hours.

13. The process of any of paragraphs 1-12, wherein fermentation product is recovered after fermentation, such as by distillation.

14. The process of any of paragraphs 1-13, wherein the fermentation product is an alcohol, preferably ethanol, especially fuel ethanol, potable ethanol and/or industrial ethanol.

15. The process of any of paragraphs 1-15, wherein the starch-containing starting material is whole grains.

16. The process of any of paragraphs 1-16, wherein the starch-containing material is derived from corn, wheat, barley, rye, milo, sago, cassava, manioc, tapioca, sorghum, rice or potatoes.

17. The process of any of paragraphs 1-17, wherein the fermenting organism is yeast, preferably a strain of Saccharomyces, especially a strain of Saccharomyces cerevisiae.

18. The process of any of paragraphs 1-18, wherein the alpha-amylase is a bacterial alpha-amylase, wherein the bacterial alpha-amylase is derived from the genus Bacillus, such as a strain of Bacillus stearothermophilus, in particular a variant of a Bacillus stearothermophilus alpha-amylase, such as the one shown in SEQ ID NO: 1 herein, in particular a truncated Bacillus stearothermophilus alpha-amylase, preferably having from 485-495 amino acids, such as around 491 amino acids.

19. The process of any paragraph 18, wherein the Bacillus stearothermophilus alpha-amylase is the one shown as SEQ ID NO: 1 herein or having sequence identity to SEQ ID NO: 1 of at least 60 percent, at least 70 percent, at least 80 percent, at least 90 percent, at least 95 percent, at least 96 percent, at least 97 percent, at least 98 percent, at least 99 percent.

20. The process of paragraph 18 or 19, wherein the Bacillus stearothermophiius alpha-amylase has one or more of the following sets of mutations:
I181ss+G182ss;
I181ss+G182ss-^{H}N193F;
   preferably —I181ssH-G182ss+E129V+K177LH-R179E;
I181ss+G182ss+N193F+E129V+K177L+R179E;
I181ss+G182ss+N193F+V59A+Q89R+E129V+177L+R179E+H208Y+K220P+N224L+Q254S
I181ss+G182ss+N193F+V59A Q89R+E129V+K177L+R179E+Q254S+IV1284V; and —
I181ss+G182ss+N193F+E129V+K177LH-R179E+K220P+N224L+S242Q+Q254S (using SEQ ID NO: 1 for numbering).

21. The process of any of paragraphs 18 to 21, wherein the Bacillus stearothermophilus alpha-amylase variant has a sequence identity to SEQ ID NO: 1 of at least 60 percent, at least 70 percent, at least 80 percent, at least 90 percent, at least 95 percent, at least 96 percent, at least 97 percent, at least 98 percent, at least 99 percent, but less than 100 percent.

22. The process of any one of paragraphs 1 to 21, wherein a protease is present and/or added during liquefaction step (a).

23. Use of a triacylglycerol lipase in a liquefaction step of fermentation product production process for increasing enzymatically accessible starch, for example, by reducing starch retrogradation.

24. Use of paragraph 23 wherein the triacylglyceride lipase has: (i) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 96 percent, at least 97 percent, at least 98 percent, at least 99 percent, such as 100 percent identity to the mature part of the polypeptide of SEQ ID NO: 3 herein, preferably derived from a strain of the genus Rhizomucor, such as a strain of Rhizomucor miehei; (ii) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 96 percent, at least 97 percent, at least 98 percent, at least 99 percent, such as 100 percent identity to the mature part of the polypeptide of SEQ ID NO: 4 herein, preferably derived from a strain of the genus Aspergillus, such as a strain of Aspergillus oryzae; (iii) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 98 percent, at least 97 percent, at least 98 percent, at least 99 percent, such as 100 percent identity to the mature part of the polypeptide of SEQ ID NO: 5 or SEQ ID NO: 6 herein, preferably derived from a strain of the genus Moesziomyces, such as a strain of Moesziomyces antarcticus; or (iii) at least 60 percent, such as at least 70 percent, such as at least 75 percent, preferably at least 80 percent, more preferably at least 85 percent, more preferably at least 90 percent, more preferably at least 91 percent, more preferably at least 92 percent, even more preferably at least 93 percent, most preferably at least 94 percent, and even most preferably at least 95 percent, such as even at least 98 percent, at least 97 percent, at least 98 percent, at least 99 percent, such as 100 percent identity to the mature part of the polypeptide of SEQ ID NO: 7 or SEQ ID NO: 8 herein, preferably derived from a strain of the genus Thermomyces, such as a strain of Thermomyces lanuginosus.

The disclosure described and claimed herein is not to be limited in scope by the specific embodiments herein disclosed, since these embodiments are intended as illustrations of several aspects of the disclosure. Any equivalent embodiments are intended to be within the scope of this disclosure. Indeed, various modifications of the disclosure in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present disclosure is further described by the following examples which should not be construed as limiting the scope of the disclosure.

### Materials & Methods

Alpha-Amylase 369 (AA369): Bacillus stearothermophilus alpha-amylase with the mutations: I181*+G182*+N193F+V59A+Q89R+E129V+K177L+R179E+Q254S+M284V truncated to 491 amino acids (SEQ ID NO: 1 herein).

Pfu protease: Pyrococcus furiosus protease (SEQ ID NO: 2)

Rm TG lipase: Rhizomucor miehei triacylglycerol lipase (SEQ ID NO: 3 herein).

Ao TG lipase: Aspergillus oryzae triacylglycerol lipase (SEQ ID NO: 4 herein).

Ma TG lipase 1: Moesziomyces antarcticus triacylglycerol lipase (SEQ ID NO: 5 herein).

Ma TG lipase 2: Moesziomyces antarcticus triacylglycerol lipase (SEQ ID NO: 6 herein).

T/TG lipase 1: Thermomyces lanuginosus triacylglycerol lipase (SEQ ID NO: 7 herein).

T/TG lipase 2: Thermomyces lanuginosus triacylglycerol lipase (SEQ ID NO: 8 herein).

Glucoamylase SA (GSA): Blend comprising Talaromyces emersonii glucoamylase disclosed as SEQ ID NO: 34 in WO99/28448 or SEQ ID NO: 13 herein, Trametes cingulata glucoamylase disclosed as SEQ ID NO: 2 in WO 06/69289 or SEQ ID NO: 14 herein, and Rhizomucor pusillus alpha-amylase with Aspergillus niger glucoamylase linker and starch binding domain (SBD) disclosed in SEQ ID NO: 15 herein having the following substitutions G128D+D143N (activity ratio in AGU:AGU:FAU-F is about 20:5:1).

Yeast: ETHANOL RED™ available from Red Star/Lesaffre, USA.

### Determination of Td by Differential Scanning Calorimetry.

The thermostability of the lipases listed in the table below were determined at pH 5.0 by Differential Scanning calorimetry (DSC) using a VP-Capillary Differential Scanning calorimeter (MicroCal Inc., Piscataway, N.J., USA) at a protein concentration of approximately 0.5 mg/ml. The thermal denaturation temperature, Td (° C.), was taken as the top of denaturation peak (major endothermic peak) in thermograms (Cp vs. T) obtained after heating enzyme solutions in buffer at a constant programmed heating rate of 200 K/hr. Sample- and reference-solutions (approx. 0.2 ml) were loaded into the calorimeter (reference: buffer without enzyme) from storage conditions at 10° C. and thermally pre-equilibrated for 20 minutes at 20° C. prior to DSC scan from 20° C. to 100° C. Denaturation temperatures (Td) were determined at an accuracy of approximately +/−1° C. The Tds obtained under these conditions for TG lipases are shown in the table below.

| TG Lipase: | Td @ pH 5 (deg C.) | Scanrate in DSC exp (degC./Hr) |
|---|---|---|
| Rm TG lipase | 65 | 200 |
| (SEQ ID NO: 3) | | |
| Ao TG lipase | 86 | 90 |
| (SEQ ID NO: 4) | | |
| Ma TG lipase 1 | 90 | 90 |
| (SEQ ID NO: 5) | | |
| Ma TG lipase 2 | 65 | 90 |
| (SEQ ID NO: 6) | | |
| Tl TG lipase 1 | 73 | 90 |
| (SEQ ID NO: 7) | | |

### EXAMPLES

### Example 1—Use of Triacylglyceride Lipase in Liquefaction Increases Ethanol Yield

This example demonstrates that the presence of triacylglyceride lipases during the liquefaction step of a fermentation product production process increases fermentation ethanol yields, for example, by increasing enzymatically accessible starch.

### Substrate

Whole corn kernels purchased by Novozymes were ground into a fine flour. After grinding, the flour was measured to be approximately 85% dry solids.

### Enzymes

The enzymes used in this example are shown in the table below:

| Enzyme | Dose (ug/g-dry solids) |
|---|---|
| Rm TG lipase (SEQ ID NO: 3) | 100, 500 |
| Ao TG lipase (SEQ ID NO: 4) | 100, 500 |
| Ma TG lipase 1 (SEQ ID NO: 5) | 100, 500 |
| Ma TG lipase 2 (SEQ ID NO: 6) | 100, 500 |
| Tl TG lipase 1 (SEQ ID NO: 7) | 100, 500 |

### Liquefaction Procedure

Flour was weighed into Lab-O-Mat canisters. Tap water was added to the flour to make a slurry at approximately 36% dry solids. The slurry was pH adjusted to pH 5.0 to pH 5.5. A commercial alpha-amylase cocktail comprising Alpha-Amylase 369 (AA369) and Pfu protease was added at an industry relevant dose. TG lipase treatments were added on top of the amylase dose. Samples were liquefied in the Lab-O-Mat at 85° C. for 2 hours. When the reaction was completed, the canisters were placed on ice and refrigerated. The material was stored frozen until use.

### Fermentation Procedure

Liquified mash treatments were fermented at a 5 g scale. Mashes were pH adjusted to approximately pH 5.0. Exogenous nitrogen in the form of urea was added, along with antibiotic penicillin. The adjusted mash was weighed into 15 mL tubes and additional tap water was added to bring the % dry solids to approximately 20% or 32%. A commercial glucoamylase blend, Glucoamylase SA (GSA), was added at an industry relevant dose. Ethanol Red ADY (activated dry yeast) was added at a 1 g/L pitch. The treatments fermented at 32° C. for approximately 50+ hours.

### HPLC Analysis

Fermentations were sampled at either ˜24 hours or ˜50 hours to examine soluble carbohydrates and organic acids. Samples were first acidified with 40% H2SO4 to stop the reaction and then centrifuged at approximately 3 krpm for up to 5 minutes. The supernatant was then filtered through a 0.2 um filter. The filtrate was then measured via HPLC using an H-column. Analytes of interest were: DP4, DP3, glucose, fructose, arabinose, lactic acid, glycerol, acetic acid, and ethanol.

### Data Analysis

Data was analyzed using SAS JMP statistical software.

### Results

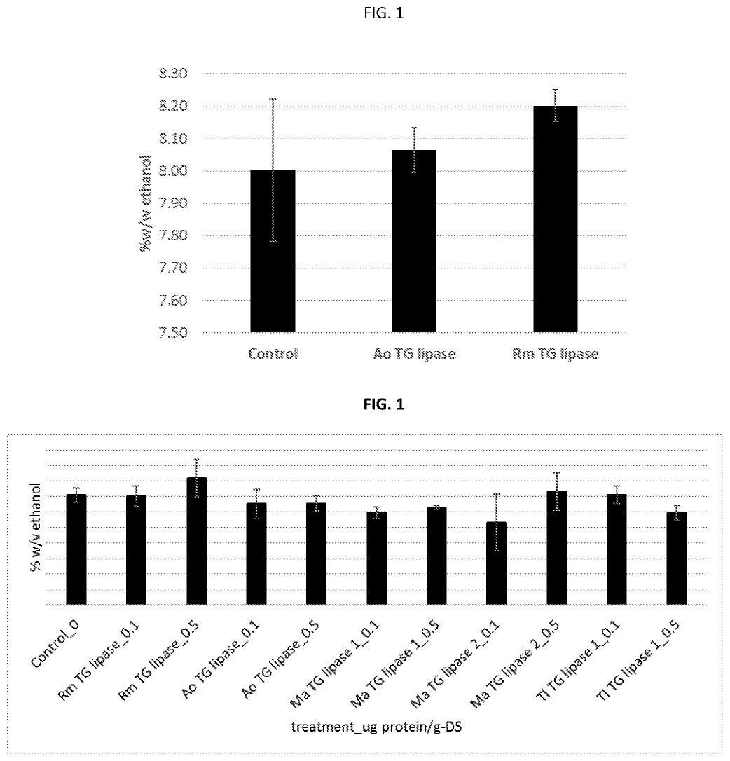

FIG. 1 is a graph depicting the results from primary screening at 20% dry solids (DS) at a 24 hr time point, showing that Rm TG lipase and Ao TG lipase improved ethanol titers compared to the control treatment lacking a TG lipase.

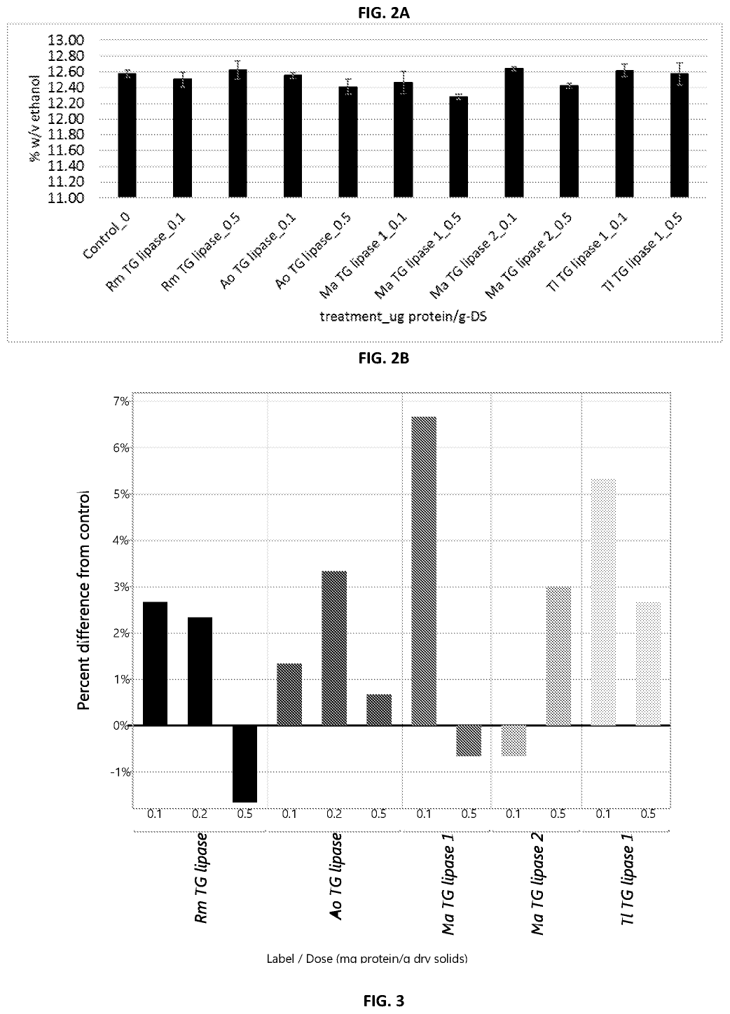

FIG. 2A is a graph depicting the results from secondary screening at 32% DS at a 24 hr time point, showing the affect of TG lipases on ethanol titers compared to the control treatment.

FIG. 2B is a graph depicting the results from secondary screening at 32% DS at a 60 hr time point, showing the affect of TG lipases on ethanol titers compared to the control treatment.

The final ethanol and percentage of ethanol increase by addition of TG lipase is summarized in Table 2 below.

TABLE 2
Summarized Ethanol Yield and Percent Change Results

| | 24 hours | | 60 hours | |
|---|---|---|---|---|
| | | Ethanol | | Ethanol |
| | (% w/v) | Increase | (% w/v) | Increase |
| Treatment | Ethanol | (%) | Ethanol | (%) |
| AA369 and Pfu control | 10.22 | 0.00 | 12.57 | 0.00 |
| AA369 and PFU control plu | 10.20 | −0.01 | 12.50 | −0.07 |
| 100 μg of Rm TG lipase (SEQ ID | | | | |
| NO: 3) | | | | |
| AA369 and PFU control plu | 10.44 | 0.22 | 12.62 | 0.05 |
| 500 μg of Rm TG lipase (SEQ ID | | | | |
| NO: 3) | | | | |
| AA369 and Pfu control plus | 10.10 | −0.11 | 12.55 | −0.02 |
| 100 μg of Ao TG lipase (SEQ ID | | | | |
| NO: 4) | | | | |
| AA369 and Pfu control plus | 10.11 | −0.11 | 12.41 | −0.17 |
| 500 μg of Ao TG lipase (SEQ ID | | | | |
| NO: 4) | | | | |
| AA369 and Pfu control plus | 9.99 | −0.23 | 12.46 | −0.11 |
| 100 μg of Ma TG lipase 1 (SEQ | | | | |
| ID NO: 5) | | | | |
| AA369 and Pfu control plus | 10.06 | −0.16 | 12.28 | −0.29 |
| 500 μg of Ma TG lipase 1 (SEQ | | | | |
| ID NO: 5) | | | | |
| AA369 and Pfu control plus | 9.87 | −0.35 | 12.64 | 0.07 |
| 100 μg of Ma TG lipase 2 (SEQ | | | | |
| ID NO: 6) | | | | |
| AA369 and Pfu control plus | 10.26 | 0.04 | 12.42 | −0.15 |
| 500 μg of Ma TG lipase 2 (SEQ | | | | |
| ID NO: 6) | | | | |
| AA369 and Pfu control plus | 10.22 | 0.00 | 12.61 | 0.04 |
| 100 μg of Tl TG lipase 1 (SEQ ID | | | | |
| NO: 7) | | | | |
| AA369 and Pfu control plus | 9.99 | −0.23 | 12.57 | 0.00 |
| 500 μg of Tl TG lipase 1 (SEQ ID | | | | |
| NO: 7) | | | | |

FIG. 3 is a graph depicting the results from incubating liquified mash samples with Alpha-Amylase and Glucoamylase, showing an increase in the amount of enzymatically accessible starch after TG lipase treatment for all lipases tested. The effect of lipase treatment is dose dependent. Some doses have a negative impact. The effective dose depends on the type of lipase employed.

### Example 2—Use of a Triacylglyceride Lipase in SSF to Increase Ethanol Yield

A control mash was prepared in-house with an industry relevant doses of AA369 and Pfu protease using a Lab-O-Mat incubator for 2 hours at 85° C. and 36% DS to simulate typical industry conditions. The mash was then frozen prior to use in SSF. For SSF, all mash was prepared with 1000 ppm of urea and 3 ppm of penicillin to aid with yeast fermentation and mitigate potential contaminants. All treatments were dosed with a baseline commercial glucoamylase blend (GSA), while TG lipase treatments were dosed on top at 1600 ug/g-DS. SSF was performed at 5 g scale with 1 g/L Ethanol Red yeast at 32° C. for up to 60 hours at 32% DS. At the end of fermentation, samples were deactivated with 50 uL of 40% sulfuric acid and then centrifuged. The supernatant was filtered through a 0.2 um filter and then measured for soluble carbohydrates, alcohols and organic acids using an ion-exchange H-column on HPLC.

Unexpectedly, as shown in Table 3 below, only Rm TG lipase show an ethanol increase by addition to simultaneous saccharification and fermentation (SSF), while all other treatments saw a decrease in ethanol titers. The dosing of TG lipases in this case is far above industry relevant dosing.

### Results:

TABLE 3
Percent Ethanol Increase After TG Lipase Addition to SSF

| | | Ethanol | Percent |
|---|---|---|---|
| | (% w/v) | Increase | Ethanol |
| Treatment | Ethanol | (%) | Increase |
| AA369 and Pfu control | 13.13 | 0.00 | |
| AA369 and PFU control plu 1600 μg of | 13.36 | 0.22 | 1.8% |
| Rm TG lipase (SEQ ID NO: 3) | | | |
| AA369 and Pfu control plus 1600 μg of | 12.94 | −0.19 | −1.4% |
| Ao TG lipase (SEQ ID NO: 4) | | | |
| AA369 and Pfu control plus 1600 μg of | 12.65 | −0.48 | −3.7% |
| Ma TG lipase 1 (SEQ ID NO: 5) | | | |
| AA369 and Pfu control plus 1600 μg of | 13.09 | −0.04 | −0.3% |
| Ma TG lipase 2 (SEQ ID NO: 6) | | | |
| AA369 and Pfu control plus 1600 μg of | 13.03 | −0.10 | −0.8% |
| Tl TG lipase 1 (SEQ ID NO: 7) | | | |

1. A process for increasing enzymatically accessible starch and/or increasing fermentation product yield, such as especially ethanol, during a fermentation product production process, wherein a triacylglycerol lipase is present and/or added before or during a liquefaction step and/or before or during a saccharification step, a fermentation step, or a simultaneous saccharification and fermentation step of the fermentation product production process.

2. A process for producing a fermentation product, comprising the steps of:
(a) liquefying a starch-containing material using an alpha-amylase;
(b) saccharifying the liquefied starch-containing material using a carbohydrate-source generating enzyme to form fermentable sugars; and
(c) fermenting the fermentable sugars using a fermenting organism to product the fermentation product, wherein a triacylglycerol lipase is added before or during liquefying step (a) and/or added before or during saccharifying step (b), fermenting step (c) or simultaneous saccharification and fermentation.

3. The process of claim 2, wherein the triacylglycerol lipase is a thermostable triacylglycerol lipase, having a Melting Point (DSC) between 60° C. and 110° C., between 65° C. and 95° C., between 70° C. and 90° C., above 70° C., above 72° C., above 80° C., above 85° C., above 90° C., above 92° C., above 94° C., above 96° C., above 98° C., above 100° C.

4. The process of claim 2, wherein the triacylglycerol lipase has: (i) at least 80 percent, at least 85 percent, at least 90 percent, at least 91 percent, at least 92 percent, at least 93 percent, at least 94 percent, at least 95 percent, at least 96 percent, at least 97 percent, at least 98 percent, at least 99 percent, or at least 100 percent identity to the mature part of the polypeptide of SEQ ID NO: 3 herein; (ii) at least 80 percent, at least 85 percent, at least 90 percent, at least 91 percent, at least 92 percent, at least 93 percent, at least 94 percent, at least 95 percent, at least 96 percent, at least 97 percent, at least 98 percent, at least 99 percent, or at least 100 percent identity to the mature part of the polypeptide of SEQ ID NO: 4 herein; (iii) at least 80 percent, at least 85 percent, at least 90 percent, at least 91 percent, more preferably at least 92 percent, even more preferably at least 93 percent, at least 94 percent, at least 95 percent, at least 98 percent, at least 97 percent, at least 98 percent, at least 99 percent, or at least 100 percent identity to the mature part of the polypeptide of SEQ ID NO: 5 or SEQ ID NO: 6 herein; or (iii) at least 80 percent, at least 85 percent, at least 90 percent, at least 91 percent, at least 92 percent, at least 93 percent, at least 94 percent, least 95 percent, at least 98 percent, at least 97 percent, at least 98 percent, at least 99 percent, or at least 100 percent identity to the mature part of the polypeptide of SEQ ID NO: 7 or SEQ ID NO: 8 herein.

5. The process of claim 2, wherein an alpha-amylase and a triacylglyceride lipase having a Melting Point (DSC) above 72 degrees centigrade, above 80 degrees centigrade, above 82 degrees centigrade, at least 86 degrees centigrade, or 90 degrees centigrade, are present and/or added in liquefaction step (a), saccharification step (b), simultaneous saccharification and fermentation, or after step (a) and before step (b) during pre-saccharification.

6. The process of claim 2, wherein saccharification and fermentation is carried out sequentially or simultaneously.

7. The process of claim 2, wherein the fermentation product is an alcohol, preferably ethanol, especially fuel ethanol, potable ethanol and/or industrial ethanol.

8. The process of claim 2, wherein the starch-containing starting material is whole grains.

9. The process of claim 2, wherein the starch-containing material is derived from corn, wheat, barley, rye, milo, sago, cassava, manioc, tapioca, sorghum, rice or potatoes.

10. The process of claim 2, wherein the fermenting organism is a yeast strain of the genus Saccharomyces.

11. The process of claim 2, wherein the alpha-amylase is a bacterial alpha-amylase, wherein the bacterial alpha-amylase is derived from the genus Bacillus.

12. The process of any claim 2, wherein the alpha-amylase is the one shown as SEQ ID NO: 1 herein or one having a sequence identity to SEQ ID NO: 1 of at least 80 percent, at least 90 percent, at least 95 percent, at least 96 percent, at least 97 percent, at least 98 percent, or at least 99 percent.

13. The process of claim 12, wherein the alpha-amylase has one or more of the following sets of mutations:
I181ss+G182ss;
I181ss+G182ss-^{H}N193F;
preferably —I181ssH-G182ss+E129V+K177LH-R179E;
I181ss+G182ss+N193F+E129V+K177L+R179E;
I181^{ss}+G182ss+N193F+V59A+Q89R+E129V+177L+R179E+H208Y+K220P+N224L+Q254S
I181^{ss}+G182ss+N193F+V59A Q89R+E129V+K177L÷R179E+Q254S+IVI284V; and—
I181ss+G182ss+N193F+E129V+K177LH-R179E+K220P+N224L+S242Q+Q254S (using SEQ ID NO: 1 for numbering).

14. The process of claim 13, wherein the alpha-amylase has a sequence identity to SEQ ID NO: 1 of at least 80 percent, at least 90 percent, at least 95 percent, at least 96 percent, at least 97 percent, at least 98 percent, or at least 99 percent, but less than 100 percent.

15. The process of claim 2, wherein a protease is present and/or added during liquefaction step (a).

16. (canceled)

17. (canceled)
